(19) United States
(12) Patent Application Publication
(10) Pub. No.: US 2021/0275547 A1
(43) Pub. Date: Sep. 9, 2021

(54) Methods of Modulating Activity of a Cyclic Dinucleotide (CDN) with a CDN Transporter-Modulating Agent

(71) Applicant: THE REGENTS OF THE UNIVERSITY OF CALIFORNIA, Oakland, CA (US)

(72) Inventors: David H. RAULET, Berkeley, CA (US); Rutger D. LUTEIJN, Albany, CA (US); Jacob E. CORN, ZURICH (CH)

(73) Assignee: The Regents of the University of California, Oakland, CA (US)

(21) Appl. No.: 17/053,963

(22) PCT Filed: May 16, 2019
(86) PCT No.: PCT/US2019/032663

§ 371 Date: Nov. 9, 2020

Related U.S. Application Data
(60) Provisional application No. 62/702,242, filed on Jul. 23, 2018.
(60) Provisional application No. 62/673,073, filed on May 17, 2018.

(51) Int. Cl. A61K 31/635; A61K 31/7084

(52) U.S. Cl. CPC A61K 31/635; A61K 31/7084

(57) ABSTRACT

Methods of modulating the activity of a cyclic dinucleotide (CDN) in a cell via membrane transporter are provided. Aspects of the methods may include contacting a cell with a CDN transporter-modulating agent to modulate transport of a CDN into the cell. In some cases, the CDN transporter-modulating agent modulates SLC19A1-mediated transport of the CDN into the cell. Also provided are compositions and kits for use in practicing the subject methods. The methods and compositions find use in a variety of applications, including therapeutic applications, such as methods of treating cancer or an inflammatory disease.

### CROSS-REFERENCE TO RELATED APPLICATIONS

Pursuant to 35 U.S.C. § 119(e), this application claims priority to U.S. Provisional Application Ser. No. 62/673,073 filed on May 17, 2018 and U.S. Provisional Application Ser. No. 62/702,242 filed Jul. 23, 2018, the disclosures of which are herein incorporated by reference.

### INTRODUCTION

The innate immune system, once activated, initiates broader immune responses mediated by T cells, B cells and NK cells. The accumulation of DNA in the cytosol of infected, cancerous or mutant cells can trigger an innate immune response via the cGAS/STING pathway. The response is initiated by the binding of cytosolic DNA to the cytosolic enzyme cGAMP synthase (cGAS), leading to the synthesis of the second messenger 2′3′-cyclic GMP-AMP (2′3′-cGAMP). 2′3′-cGAMP activates the protein ‘stimulator of interferon genes’ (STING), which in turn activates the transcription factors IRF3 and NF-κB, and consequently the production of cytokines, including type I interferons that support a broader immune response.

The cGAS/STING pathway senses cytosolic DNA originating from viruses and bacteria. STING is also activated by cytosolic self-DNA, which accumulates in cells in certain autoinflammatory disorders, including Aicardi-Goutieres Syndrome and systemic lupus erythematosus. Furthermore, cytosolic DNA accumulates in cells subjected to DNA damage, as occurs in tumor cells, resulting in activation of the cGAS/STING pathway and the initiation of an anti-tumor immune response.

The natural anti-tumor immune response can be weak. An amplified anti-tumor immune response can occur when STING agonists, such as cyclic dinucleotides (CDNs), are injected into the tumor microenvironment, leading to immune activation and tumor regression.

### SUMMARY

Methods of modulating the activity of a cyclic dinucleotide (CDN) in a cell a via membrane transporter are provided. Aspects of the methods may include contacting a cell with a CDN transporter-modulating agent to modulate transport of a CDN into the cell. In some embodiments, the CDN transporter-modulating agent modulates solute carrier family 19, member 1 (SLC19A1)-mediated transport of the CDN into the cell. In some cases, the CDN transporter-modulating agent modulates solute carrier family 46, member 1 or 3, (SLC46A1 or SLC46A3)-mediated transport of the CDN into the cell. Also provided are compositions and kits for use in practicing the methods. The methods and compositions find use in a variety of applications, including therapeutic applications, such as methods of treating cancer or an inflammatory disease.

### BRIEF DESCRIPTION OF THE DRAWINGS

The skilled artisan will understand that the drawings, described below, are for illustration purposes only. The drawings are not intended to limit the scope of the present teachings in any way.

FIG. 1, panels A-E, illustrate results related to the screening and validation of CDN transporters in THP-1 cells expressing an ISRE-driven tdTomato reporter. Further details are provided in the experimental section below.

FIG. 2, panels A-B show the results of cell stimulation (either inhibition or induction) with synthetic CDN in the presence of high (panel A) or low concentrations (panel B) of sulfasalazine (SSZ). FIG. 2, panels C-D: THP-1 cells were incubated with increasing concentrations of the competitive inhibitors methotrexate (left panel), 5-methyl tetrahydrofolate (5-methyl THF, right panel) or DMSO as vehicle control, before stimulating with 2′3′-RR CDA (1.25 g/ml), 2′3′-cGAMP (15 g/ml) or hIFN- (100 ng/ml). After 18 h, tdTomato reporter expression was analyzed by flow cytometry. For each stimulant, the data were normalized to the DMSO controls.

FIG. 3, panels A-B show that overexpression of SLC46A1 increases the responses of cells to CDNs (Panel A), and that decreasing the expression of SLC46A3 decreases the response (Panel B).

### DEFINITIONS

The following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

The term “administration” or “administering” as used herein with regard to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell. By “administered together” it is not meant to be implied that two or more agents be administered as a single composition. Although administration as a single composition is contemplated by the present disclosure, such agents may be delivered to a single subject as separate administrations, which may be at the same or different time, and which may be by the same route or different routes of administration.

The term “affinity” refers to the equilibrium constant for the reversible binding of two agents; “affinity” can be expressed as a dissociation constant (Kd).

The term “binding” refers to a direct association between two molecules, due to, for example, covalent, electrostatic, hydrophobic, and ionic and/or hydrogen-bond interactions, including interactions such as salt bridges and water bridges.

The terms “cell uptake” and “cellular uptake” are used interchangeably herein and refer to the movement of a compound from the extracellular environment or matrix and into a cell, e.g., to the cytoplasm of a cell.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The terms “subject”, “individual” and “patient” are used interchangeably and refer to a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

“Mammal” means a member or members of any mammalian species, and includes, by way of example, canines; felines; equines; bovines; ovines; rodentia, etc. and primates, particularly humans. Non-human animal models, particularly mammals, e.g., primate, murine, lagomorpha, etc. may be used for experimental investigations.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. As used herein, the term “treating” is thus used to refer to both prevention of disease, and treatment of pre-existing conditions. For example, where the cyclic-di-nucleotide active agent is administered, the prevention of cellular proliferation can be accomplished by administration of the subject compounds prior to development of overt disease, e.g., to prevent the regrowth of tumors, prevent metastatic growth, etc. Alternatively, the compounds are used to treat ongoing disease, by stabilizing or improving the clinical symptoms of the patient.

Other definitions of terms may appear throughout the specification.

### DETAILED DESCRIPTION

As summarized above, methods of modulating the activity of a cyclic dinucleotide (CDN) in a cell via a membrane transporter are provided. Aspects of the methods may include contacting a cell with a CDN transporter-modulating agent to modulate transport of a CDN into the cell. In some embodiments, the CDN transporter-modulating agent modulates SLC19A1-mediated transport of the CDN into the cell. In some cases, the CDN transporter-modulating agent modulates SLC46A1 or SLC46A3-mediated transport of the CDN into the cell. Also provided are compositions and kits for use in practicing the subject methods. The methods and compositions find use in a variety of applications, including therapeutic applications, such as methods of treating cancer or an inflammatory disease.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods of Modulating Activity of a Cyclic Dinucleotide

As summarized above, methods of modulating the activity of a cyclic dinucleotide (CDN) in a cell by modulating the transport of the CDN into the cell via a membrane transporter are provided. Aspects of the subject methods include use of a CDN transporter-modulating agent to modulate the cellular uptake of the CDN via the membrane transporter of interest.

In some instances, modulating the activity of a CDN means increasing or enhancing the activity of a CDN in a cell, in vitro or in vivo, by increasing the transport of the CDN into the cell via a membrane transporter. When the cellular uptake of the CDN is increased, one or more activities of a CDN of interest can also be increased or enhanced. In certain instances, activities of a CDN that are increased or enhanced include, but are not limited to, production of type I interferon (IFN), e.g., to provide an anti-tumor immune response or an immune response against a pathogen, and intercellular 2′3′-cGAMP signaling, e.g., between virus-infected cells and uninfected cells or between tumor cells and non-tumor cells. In certain cases, the parameter of interest, e.g., production of a type I interferon in a cell, or a 2′3′-cGAMP signal, is increased or enhanced by 10% or more, such as 20% or more, 30% or more, 40% or more, 50% or more, 60% or more, 70% or more, 80% or more, 90% or more, 100% or more, 2-fold or more, 3-fold or more, or even more, e.g., relative to a control not contacted with the CDN transporter-modulating agent.

Modulating the activity of a CDN is meant to encompass enhancing the treatment of a pathologic or disease condition in which the CDN finds use, e.g., relative to treatment in the absence of the CDN transporter modulating agent. Pathologic or disease conditions of interest are described herein and include, but are not limited to, cellular proliferative disease, cancer, autoimmune or inflammatory disease, viral infection (e.g., hepatitis virus), infections with intracellular bacteria and parasites. Enhancing the treatment of a pathologic or disease condition may include amelioration of the symptoms of a particular condition, arresting or reducing the development of the disease or its symptoms, and/or stabilizing or improving the clinical symptoms of the patient. Modulating the activity of a CDN is also meant to encompass treatment of a pathologic or disease condition with an effective amount of a CDN that is reduced relative to the amount of the CDN that would otherwise be utilized as effective in the absence of the CDN transporter modulating agent.

In certain instances, modulating the activity of a CDN means decreasing or inhibiting the activity of a CDN in a cell, in vitro or in vivo, by inhibiting the transport of the CDN into the cytosol of the cell via the membrane transporter. When the cellular uptake of the CDN is decreased, one or more activities of a CDN of interest can also be decreased or inhibited. Modulating the activity of a CDN in a cell is meant to encompass ameliorating undesirable side effects of a CDN therapy for a pathologic or disease condition, e.g., relative to CDN therapy in the absence of the CDN transporter modulating agent. In certain instances, activities of a CDN that are decreased or inhibited include, but are not limited to, intercellular 2′3′-cGAMP signaling and cell toxicity. In certain cases, the parameter of interest is decreased or inhibited by 10% or more, such as 20% or more, 30% or more, 40% or more, 50% or more, 60% or more, 70% or more, 80% or more, 90% or more, 100% or more, 2-fold or more, 3-fold or more, or even more, e.g., relative to a control not contacted with the CDN transporter-modulating agent.

### CDN Transporters and CDN Transporter-Modulating Agents

As summarized above, aspects of the subject methods include use of agents that modulate cellular uptake of CDNs via membrane transporters of interest, also referred to herein as CDN transporters. Membrane transporters that can be targeted to modulate cellular uptake of a CDN of interest according to the subject methods include membrane folate transporters that are capable of transporting CDNs of interest into cells. Membrane folate transporters include a class of transporters which can actively transport molecules including folate, folate derivatives and/or antifolates, see e.g., Matherly et al. (“Membrane transport of folates”, Vitam. Horm. 2003; 66:403-56). Membrane folate transporters of interest include, but are not limited to, the SLC19 family of transporters, reduced folate carrier (RFC), the SLC46 family of transporters including the proton-coupled folate transporters (PCFT). RFC transporter is ubiquitously expressed and can transport folate in mammalian cells and tissues.

In some instances of the method, the transporter is a member of the SLC19 family of transporters. In some instances of the method, the transporter is RFC transporter. In certain cases, the RFC transporter is RFC1, also known as solute carrier family 19 (folate transporter), member 1, also known as SLC19A1, RFC, CHMD, FOLT, IFC1, REFC or IFC-1. In some instances of the method, the transporter is a member of the SLC46 family of transporters. In certain cases, the transporter is solute carrier family 46, member 1, also known as SLC46A1, PCFT, G21 or HCP1. In certain cases, the transporter is solute carrier family 46, member 3, also known as SLC46A3 or FKSG16. Exemplary transporters of interest include those described by Hou and Matherly (“Biology of the Major Facilitative Folate Transporters SLC19A1 and SLC46A1”, Curr Top Membr. 2014; 73: 175-204), Zhao and Goldman (Folate and Thiamine Transporters mediated by Facilitative Carriers (SLC19A1-3 and SLC46A1) and Folate Receptors) Mol. Aspects Med. 2013; 34) and Hamblett et al. (“SLC46A3 Is Required to Transport Catabolites of Noncleavable Antibody Maytansine Conjugates from the Lysosome to the Cytoplasm”, Cancer Res. 2015 Dec. 15; 75(24):5329-40).

Aspects of the subject methods include contacting a cell with a CDN transporter-modulating agent to modulate the transport of the CDN across the membrane of a cell thereby modulating the activity of the CDN of interest in the cell. A CDN transporter-modulating agent is an agent that is capable of modulating the action of a target membrane transporter either directly (e.g., via direct binding to produce an enhancing or inhibiting effect) or indirectly (e.g., via modulating expression of a membrane transporter). Any convenient agents that are capable of modulating the activity of a target membrane transporter can be adapted for use in the subject methods. In some instances, the agent directly binds to the target membrane transporter to modulate its activity. In certain instances, the agent acts indirectly, e.g., via modulating expression of the target membrane transporter.

CDN transporter-modulating agents of interest include, but are not limited to, a ligand, a receptor, a CDN transporter-binding antibody, a scaffolded protein binder of a CDN transporter, a nucleic acid, a small molecule, an organic anion, an inorganic ion or salt, and a peptide; or a fragment, variant, or derivative thereof; or combinations of any of the foregoing. CDN transporter-modulating agents include small molecule compounds that selectively inhibit the activity of the membrane transporter of interest. CDN transporter-modulating agents include small molecules that selectively enhance the activity of the membrane transporter. Small molecule compounds that specifically and directly bind to the membrane transporter are of interest. Naturally occurring or synthetic small molecule compounds of interest include numerous chemical classes, such as organic molecules, e.g., small organic compounds having a molecular weight of more than 50 and less than about 2,500 daltons. The compounds can include functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups.

CDN transporter-modulating agents are also found among biomolecules including proteins, peptides, saccharides, fatty acids, steroids, purines, pyrimidines, derivatives, structural analogs or combinations thereof. Such molecules may be identified using any convenient methods. In some cases, useful CDN transporter-modulating agents exhibit an affinity (Kd) for a target CDN transporter, such as SLC19A1, that is sufficient to provide for the desired modulation of CDN transport into the cell. The affinity of the CDN transporter-modulating agent can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of the agent for unrelated transporter. In some cases, the affinity of a CDN transporter-modulating agents to a target CDN transporter, e.g., a protein component of the CDN transporter can be, for example, from about 100 nanomolar (nM) to about 1 nM, from about 100 nanomolar (nM) to about 0.1 nM, from about 100 nM to about 1 picomolar (pM), or from about 100 nM to about 1 femtomolar (fM), or from about 10 nanomolar (nM) to about 0.1 nM. In some embodiments, the affinity between the agent and a target CDN transporter is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

CDN transporter-modulating agents include antibodies that specifically bind to a membrane transporter protein. In some cases, the antibody specifically binds an epitope of the membrane transporter protein that provides for inhibition of the function of the transporter. In certain cases, the antibody specifically binds a distinct epitope of the membrane transporter protein that provides for enhancement of the transport of a CDN across the membrane of a cell.

Antibodies that can be used as CDN transporter-modulating agents in connection with the present disclosure can encompass, but are not limited to, monoclonal antibodies, polyclonal antibodies, bispecific antibodies, Fab antibody fragments, F(ab)₂ antibody fragments, Fv antibody fragments (e.g., VH or VL), single chain Fv antibody fragments and dsFv antibody fragments.

Furthermore, the antibody molecules can be fully human antibodies, humanized antibodies, or chimeric antibodies. The antibodies that can be used in connection with the present disclosure can include any antibody variable region, mature or unprocessed, linked to any immunoglobulin constant region. Minor variations in the amino acid sequences of antibodies or immunoglobulin molecules are encompassed by the present disclosure, providing that the variations in the amino acid sequence maintain 75% or more, e.g., 80% or more, 90% or more, 95% or more, or 99% or more of the sequence. In particular, conservative amino acid replacements are contemplated. Conservative replacements are those that take place within a family of amino acids that are related in their side chains. Whether an amino acid change results in a functional peptide can be determined by assaying the specific activity of the polypeptide derivative.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen-combining sites and is still capable of cross-linking antigen.

Antibodies that can be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules can be fully human antibodies, humanized antibodies, or chimeric antibodies. In some embodiments, the antibody molecules are monoclonal, fully human antibodies. The antibodies that can be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

In some cases, the CDN transporter-modulating agent is an antibody. In certain cases, the CDN transporter-modulating agent is an antibody fragment or binding derivative thereof. The antibody fragment or binding derivative thereof can be selected from a Fab fragment, a F(ab′)2 fragment, a scFv, a diabody and a triabody.

In some embodiments, the CDN transporter-modulating agent is a scaffolded polypeptide binder to a CDN transporter. A scaffold refers to an underlying peptidic framework (e.g., a consensus sequence or structural motif) from which a polypeptide agent arose, e.g., via phage display screening of a polypeptide library, or from a chimeric protein construct. The underlying scaffold sequence includes those residues that are fixed and variant residues that can confer on the resulting polypeptide agent's different functions, such as specific binding to a target receptor. Such structural motifs can be characterized and compared structurally as a combination of particular secondary and tertiary structural elements, or alternatively, as a comparable primary sequence of amino acid residues. Any convenient scaffolds and scaffolded polypeptides can be utilized as agents in the subject methods. In some embodiments, such agents can be identified utilizing a recombinant screening method such as phage display screening. Scaffolded polypeptide binders of interest include, but are not limited to, synthetic small proteins and recombinant small proteins such as Affibodies.

In some cases, the CDN transporter-modulating agent modulates a transporter of folate or a folate derivative. In certain instances, the CDN transporter-modulating agent modulates CDN transport via a member of the SLC19 family of transporters. In some embodiments, the CDN transporter-modulating agent modulates SLC19A1-mediated transport of the CDN into the cell. Any convenient agents directed to modulation of the action of a transporter of the SLC19 family, such as SLC19A1, can be adapted for use in the subject methods. The transport activity of folate by SLC19A1 can be altered by a variety of organic and inorganic molecules (see e.g. Jansen, G. et al. “Sulfasalazine is a potent inhibitor of the reduced folate carrier: Implications for combination therapies with methotrexate in rheumatoid arthritis.” Arthritis Rheum. 50, 2130-2139 (2004) and Goldman, “The characteristics of the membrane transport of amethopterin and the naturally occurring folates.” Ann. N. Y. Acad. Sci. 186, 400-22 (1971)), any one of which can be adapted for use as a CDN transporter-modulating agent in the subject methods. Specific inhibitors of interest include, but are not limited to: sulfasalazine, methotrexate, 5-methyltetrahydrofolate and the like.

In certain embodiments, the CDN transporter-modulating agent is sulfasalazine or a salt thereof. Sulfasalazine can be described by the following structure that includes a basic pyridyl group and an acidic salicyclic acid group:

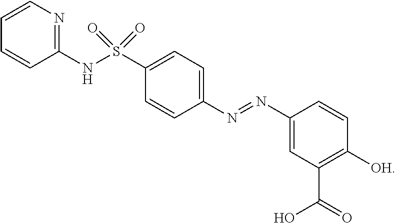 [SMILES: O=C(O)c1cc(/N=N/c2ccc(S(=O)(=O)Nc3ccccn3)cc2)ccc1O]

Due to the amphoteric nature of sulfasalazine, there is a possibility of sulfasalazine forming salts with acids as well as bases. Aspects of the present disclosure include salts of sulfasalazine, such as pharmaceutically acceptable salts.

In some instances, the agent is an antibody agent that targets a distinct epitope of the SLC19A1 protein to inhibit SLC19A1-mediated transport of CDNs. In certain instances, the agent is an antibody agent that targets a distinct epitope of the SLC19A1 protein to enhance SLC19A1-mediated transport of CDNs.

In some instances, the agent is an antibody agent that targets a distinct epitope of the SLC46A1 protein to inhibit SLC46A1-mediated transport of CDNs. In certain instances, the agent is an antibody agent that targets a distinct epitope of the SLC46A1 protein to enhance SLC46A1-mediated transport of CDNs.

In some instances, the agent is an antibody agent that targets a distinct epitope of the SLC46A3 protein to inhibit SLC46A3-mediated transport of CDNs. In certain instances, the agent is an antibody agent that targets a distinct epitope of the SLC46A3 protein to enhance SLC46A3-mediated transport of CDNs.

In some embodiments, the CDN transporter-modulating agent modulates SLC19A1-mediated transport of the CDN into the cell. In certain cases, the CDN transporter-modulating agent modulates SLC46A1-mediated transport of the CDN into the cell. In some instances, the CDN transporter-modulating agent modulates SLC46A3-mediated transport of the CDN into the cell. Any convenient agents that modulate the action of transporters SLC19A1, SLC46A3 and/or SLC46A3 can be adapted for use in the subject methods. Agents that are capable of inhibiting a membrane transporter of interest at one concentration can be adapted for use in the subject methods to provide for enhancement of transporter activity.

Depending on the particular embodiments being practiced, a variety of different types of CDN transporter-modulating agents may be employed. In some instances, the agent modulates the activity of a CDN transporter protein following expression, such that the agent is one that changes the activity of the protein encoded by the target gene following expression of the protein from the target gene. In other embodiments, the CDN transporter-modulating agent modulates expression of the RNA and/or protein from the gene encoding the CDN transporter, such that it changes the expression of the RNA or protein from the target gene in some manner. In these instances, the agent may change expression of the RNA or protein in a number of different ways. As would be readily understood by one of ordinary skill in the art, one can reduce expression (protein production) of an endogenous gene at the DNA, RNA, or protein level. For example, expression can be reduced by reducing the total amount of wild type protein made by the endogenous locus, and this can be accomplished either by changing the nature of the protein produced (e.g., via gene mutation to generate a loss of function allele such as a null allele or an allele that encodes a protein reduced function) or by reducing the overall levels of protein produced without changing the nature of the protein itself.

In certain embodiments, the CDN transporter-modulating agent is one that reduces, including inhibits, expression of a functional CDN transporter. Inhibition of protein expression may be accomplished using any convenient means, and one of ordinary skill in the art will be aware of multiple suitable methods. For example, in order to reduce/inhibit expression, one can reduce protein levels post-translationally; one can block production of protein by blocking/reducing translation of mRNA (e.g., using an RNAi agent such as an shRNA or siRNA that targets the mRNA of an endogenous gene); one can reduce mRNA levels post-transcriptionally (e.g., using an RNAi agent such as an shRNA or siRNA that targets the mRNA of an endogenous gene); one can reduce mRNA levels by blocking transcription (e.g., using gene editing tools to either alter a promoter and/or enhancer sequence or to modulate transcription, or by using modified gene editing tools, e.g., CRISPRi, that can modify transcription without cutting the target DNA). Additionally, one can alter the nature of the protein made from an endogenous locus by inducing (e.g., using gene editing technology) a loss of function mutation, which can range from an allele with reduced wild type activity to a dead protein or no protein (e.g., catalytically inactive mutant, a frameshift allele, a gene knockout, etc.). Moreover, one can reduce mRNA levels via gene editing methods that result in low net transcript levels (e.g., frameshift mutations can trigger nonsense mediated mRNA decay).

Any convenient inhibitor of expression can be utilized as an antagonist in the subject methods. Such antagonists can act to inhibit expression at a transcriptional, translational, or post-translational level. In some embodiments, the inhibitors are nucleic-acid based, including, without limitation, DNA, RNA, chimeric RNA/DNA, protein nucleic acid, and other nucleic acid derivatives. In some embodiments, the expression inhibitors encompass RNA molecules capable of inhibiting receptor production when introduced into a receptor-expressing cell (termed RNAi), including short hairpin double-stranded RNA (shRNA). In some instances, the expression inhibitors are small interfering RNA (siRNA). In some instances, the expression inhibitors are small interfering microRNA. It will be understood that any sequence capable of reducing the cell surface expression of a receptor, or reducing the expression of a receptor ligand, can be used in practicing the methods of the present disclosure.

Examples of agents that inhibit expression of an endogenous gene (.g., as described herein) include but are not limited to: (a) an RNAi agent such as an shRNA or siRNA that specifically targets mRNA encoded by the endogenous gene; (b) a genome editing agent (e.g., a Zinc finger nuclease, a TALEN, a CRISPR/Cas genome editing agent such as Cas9, Cpf1, CasX, CasY, and the like) that cleaves the target cell's genomic DNA at a locus encoding the endogenous gene (e.g., SLC19A1)—thus inducing a genome editing event (e.g., null allele, partial loss of function allele) at the locus of the endogenous gene; (c) a modified genome editing agent such as a nuclease dead zinc finger, TALE, or CRISPR/Cas nuclease fused to a transcriptional repressor protein that modulates (e.g. reduces) transcription at the locus encoding the endogenous gene (e.g., SLC19A1) (see, e.g., Qi et al., Cell. 2013 Feb. 28; 152(5):1173-83′; Gilbert et al, Cell. 2014 Oct. 23; 159(3):647-61; Larson et al., Nat Protoc. 2013 November; 8(11):2180-96).

Examples of agents that increase or activate expression of an endogenous gene (e.g., as described herein) include, but are not limited to, CRISPR activation (CRISPRa) agents.

When the agent is a CRISPR/Cas editing agent, the agent can include both the protein and guide RNA component. The guide nucleic acid (e.g., guide RNA) can be introduced into the cell as an RNA or as a DNA encoding the RNA (e.g., encoded by a DNA vector—on a plasmid, virus, and the like). The CRISPR/Cas protein can be introduced into the cell as a protein or as a nucleic acid (mRNA or DNA) encoding the protein. Programmable gene editing agents and their guide nucleic acids include, but are not limited to, CRISPR/Cas RNa-guided proteins such as Cas9, CasX, CasY, and Cpf1, Zinc finger proteins such as Zinc finger nucleases, TALE proteins such as TALENs, CRISPR/Cas guide RNAs, and the like.

For example, antisense molecules can be used to down-regulate expression of a target gene in the cell. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted protein, and inhibits expression of the targeted protein. Antisense molecules inhibit gene expression through various mechanisms, e.g., by reducing the amount of mRNA available for translation, through activation of RNAse H, or steric hindrance. One or a combination of antisense molecules may be administered, where a combination may include multiple different sequences.

Antisense oligonucleotides may be chemically synthesized by methods known in the art (see Wagner et al. (1993), supra, and Milligan et al., supra.) Oligonucleotides may be chemically modified from the native phosphodiester structure, in order to increase their intracellular stability and binding affinity. A number of such modifications have been described in the literature, which alter the chemistry of the backbone, sugars or heterocyclic bases.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). In addition, the transcription level of a protein can be regulated by gene silencing using RNAi agents, e.g., double-strand RNA (Sharp (1999) Genes and Development 13: 139-141). RNAi, such as double-stranded RNA interference (dsRNAi) or small interfering RNA (siRNA), has been extensively documented in the nematode C. elegans (Fire, A., et al, Nature, 391, 806-811, 1998) and routinely used to “knock down” genes in various systems. RNAi agents may be dsRNA or a transcriptional template of the interfering ribonucleic acid that can be used to produce dsRNA in a cell. A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

### Cyclic Dinucleotides (CDNs)

As reviewed above, methods of modulating the transport of a CDN into a cell are provided. The CDN of interest can be a CDN that is contacted with a cell in vitro or administered to a subject in vivo. As such, aspects of the subject methods include contacting a target cell with the CDN of interest. A variety of CDNs find use in the subject methods in conjunction with the CDN transporter modulating agents (e.g., as described herein).

In some cases, the CDN is naturally occurring. Naturally occurring CDNs of interest include those involved in intercellular signaling, such as 2′3′-cGAMP. In certain instances, the CDN is one that is implicated in a disease or condition associated with aberrant signaling, such as an autoimmune/inflammatory disease (e.g., as described herein). In some cases, the CDN is involved in intercellular signaling between tumor cells and non-tumor cells where amplification of the signal can provide for anti-tumor immunity. In some cases, the CDN is involved in intercellular signaling between virus-infected and uninfected cells where amplification of the signal can provide for anti-viral immunity. In some cases, the CDN of interest is a CDN that is produced endogenously in a cell sample in vitro or in vivo by a cell of a subject. As such, aspects of the subject methods include enhancing the uptake by a target cell of an endogenously produced CDN of interest. In some instances, the endogenously produced CDN is 2′3′-cGAMP. As such, aspects of the subject methods include increasing intercellular 2′3′-cGAMP signaling between cells in vivo, such as between virus-infected and uninfected cells for amplification of anti-viral immunity. In some cases, the endogenous production of a CDN of interest can be triggered or enhanced in a CDN producing cell by administration of an CDN production promoting agent, see e.g., Vance et al. in U.S. Publication No. 2014/0329889.

In certain instances, the CDN is non-naturally occurring. In some cases, the CDN is a CDN drug that finds use in cancer therapeutic applications. A variety of CDNs that are agonists of Stimulator of Interferon Genes (STING) find use in cancer immunotherapy, including synthetic CDNs that are analogues of a naturally occurring CDN such as 2′3′-cGAMP. An amplified anti-tumor immune response can occur when a CDN STING agonist is delivered to a tumor microenvironment, leading to immune activation and tumor regression.

As used herein, “cyclic dinucleotide” or “CDN” refers to a compound containing two nucleosides (i.e., a first and second nucleoside), wherein the 2′ or 3′ carbon of each nucleoside is linked to the 5′ carbon of the other nucleoside via a phosphodiester internucleoside linkage. Therefore, a 2′-5′ phosphodiester linkage containing CDN refers to a CDN where the 2′ carbon of at least one of the nucleosides is linked to the 5′ carbon of the other nucleoside. As discussed herein, 2′-5′ phosphodiester linkage containing CDNs can be used in practicing the subject methods to increase production of a type I interferon in a cell or subject. In certain embodiments, the CDN has two 2′-5′ phosphodiester linkages. In some embodiments, the CDN has a 2′-5′ phosphodiester linkage and a 3′-5′ phosphodiester linkage. In certain embodiments, the CDN has two 3′-5′ phosphodiester linkages.

Cyclic-di-nucleotides include those specifically described herein as well as isoforms (e.g., tautomers) of those specifically described herein that can be used in practicing the subject methods. A “cyclic-di-nucleotide” also includes all of the stereoisomeric forms of the cyclic-di-nucleotides described herein.

The term “nucleoside” refers to a composition containing a nitrogenous base covalently attached to a sugar (e.g., ribose or deoxyribose) or an analog thereof. Examples of nucleosides include, but are not limited to, cytidine, uridine, adenosine, guanosine, thymidine and inosine. The term “nitrogenous base” refers to a nitrogen-containing heterocycle having the chemical properties of a nucleobase. Nitrogenous bases of interest include, but are not limited to, pyrimidines (e.g., cytosine, thymine, and uracil) and purines (e.g., adenine and guanine), as well as substituted pyrimidine derivatives and substituted purine derivatives, pyrimidine analogs and purine analogs, and tautomers thereof.

In some embodiments, the nucleoside contains a deoxyribose sugar. Analogs of nucleosides include, but are not limited to dexoyadenosine analogues (e.g., Didanosine and Vidarabine); deoxycytidine analogues (e.g., Cytarabine, Ematricitabine, Lamivudine, and Zalcitabine); deoxyguanosine analogues (Abacavir and Entecavir); (deoxy-) thymidine analogues (e.g., Stavudine, Telbivudine, and Zidovudine); and deoxyuridine alaogues (e.g., Idoxuridine and Trifluridine).

The CDN can include a guanosine nucleoside. In some cases, the CDN contains two guanosine nucleosides. The CDN can include an adenosine nucleoside. In some embodiments, the CDN contains two adenosine nucleosides. In certain cases, the CDN contains an adenosine nucleoside and a guanosine nucleoside.

While not being bound by any particular theory of operation, CDNs can increase type-I IFN production in a cell. In certain embodiments, the CDN increases type-I IFN production through a mechanism that involves stimulator of interferon genes (STING). CDNs can be obtained using any suitable method. For example, CDNs may be made by chemical synthesis using nucleoside derivatives as starting material. CDNs can also be produced via in vitro synthesis, using recombinant purified cGAMP synthase (cGAS) or other recombinant purified CDN synthases such as the bacterial cGAMP synthase from V. cholerae (DncV) or mutant versions of any recombinant purified CDN synthases. Moreover, the structures of such cyclic-di-nucleotides can be confirmed using any convenient methods, such as NMR analysis.

Any convenient CDNs may be utilized in the subject methods, compositions and kits. CDN's of interest include, but are not limited to, those described by Vance et al. in U.S. Publication No. 2014/0329889; Dubensky et al. in U.S. Publication No. 2015/0056224; Dubensky et al. in U.S. Publication No. 2014/0205653; Dubensky et al. in U.S. Pat. No. 9,549,944; Altman et al. in WO2017027645; and Altman et al. in WO2017027646, US20160362441, US20180002369, US20180064745, US2018273578, US2018186828, U.S. Pat. No. 9,718,848, WO2017075477, WO2017123657, WO2017123669, WO2017161349 WO2018009466, US20180092937, WO2018065360, WO2018045204, WO2018098203, WO2018009648, WO2018009652, WO2018100558, WO2018138684, WO2018138685, WO2018156625, WO2018198076, WO2018198084, WO2018208667, WO2019023459, WO2019043634, WO2019046496, WO2019046498, WO2019046500, WO2019046511, WO2019051488 and WO2019074887, the disclosures of which are herein incorporated by reference in their entirety.

In some cases, the CDN that finds use in the subject methods is one that is described by Vance et al. in U.S. Publication No. 2014/0329889. In certain embodiments, the cyclic-di-nucleotide has one of the following formulae (I) and (II):

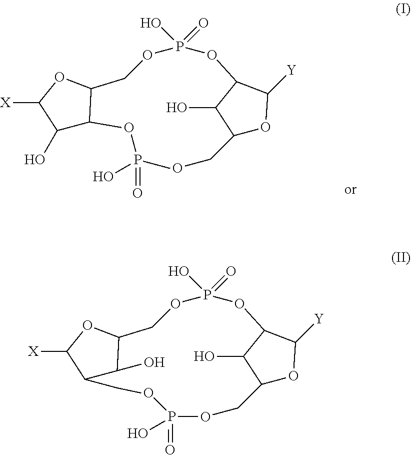

wherein X and Y are each independently a nitrogenous base or an analog thereof, or a salt thereof. In certain embodiments of formulae (I)-(II), X and Y are each independently selected from the following:

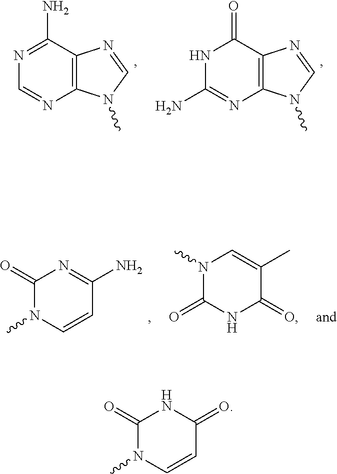

CDNs described herein can also be described by the following nomenclature: cyclic[X₁(a-5′)pX₂(b-5′)p], wherein X₁ and X₂ are first and second nucleosides, “a” is the designation of the carbon of the first nucleoside (e.g., 2′ or 3′ position) that is linked to the 5′ carbon of the second nucleoside via a phosphodiester bond and “b” is the designation of the carbon of the second nucleoside (e.g., 2′ or 3′ position) that is linked to the 5′ carbon of the first nucleoside by a phosphodiester bond. In some cases, at least one of “a” and “b” is 2′ in the formula. For instance, based on this nomenclature, cyclic[G(2′-5′)pA(3′-5′)p] has the following formula:

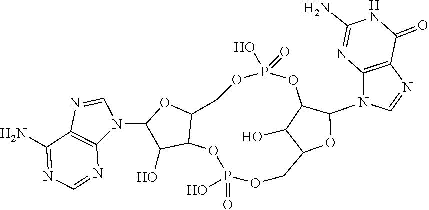 [SMILES: Nc1nc2c(ncn2C2OC3COP(=O)(O)OC4C(COP(=O)(O)OC2C3O)OC(n2cnc3c(N)ncnc32)C4O)c(=O)[nH]1]

or a salt thereof.

In certain embodiments, the CDN contains a 2′-5′ phosphodiester bond. In particular embodiments, the CDN further contains a 3′-5′ phosphodiester bond (e.g., cyclic[X₁(2′-5′)pX₂(3′-5′)p] or cyclic[X₁(3′-5′)pX₂(2′-5′)p]). In some instances, the CDN contains two 2′-5′ phosphodiester bonds (cyclic[X₁(2′-5′)pX₂(2′-5′)p]). In some instances, the CDN contains two 3′-5′ phosphodiester bonds (cyclic[X₁(3′-5′)pX₂(3′-5′)p]).

In certain embodiments, the cyclic-di-nucleotide is: cyclic[A(2′-5′)pA2′-5)p]; cyclic[T(2′-5′)pT(2′-5)p]; cyclic[G(2′-5′)pG(2′-5)p]; cyclic[C(2′-5′)pC(2′-5)p]; or cyclic[U(2′-5′)pU(2′-5′)p]. In certain embodiments, the cyclic-di-nucleotide is: cyclic[A(2′-5′)pA(3′-5)p]; cyclic[T(2′-5′)pT(3′-5)p]; cyclic[G(2′-5′)pG(3′-5)p]; cyclic[C(2′-5′)pC(3′-5)p]; cyclic[U(2′-5′)pU(3′-5′)p]; cyclic[A(2′-5′)pT(3′-5)p]; cyclic[T(2′-5′)pA(3′-5)p]; cyclic[A(2′-5′)pG(3′-5)p]; cyclic[G(2′-5′)pA(3′-5)p]; cyclic[A(2′-5′)pC (3′-5′)p]; cyclic[C(2′-5′)pA(3′-5)p]; cyclic[A(2′-5′)pU(3′-5′)p]; cyclic[U(2′-5′)pA(3′-5)p]; cyclic[T(2′-5′)pG(3′-5)p]; cyclic[G(2′-5′)pT(3′-5)p]; cyclic[T2′-5′)pC(3′-5)p]; cyclic[C(2′-5′)pT(3′-5)p]; cyclic[T(2′-5′)pU(3′-5)p]; cyclic[U(2′-5′)pT(3′-5′)p]; cyclic[G(2′-5′)pC(3′-5)p]; cyclic[C2′-5′)pG(3′-5)p]; cyclic[G(2′-5′)pU(3′-5)p]; cyclic[U(2′-5′)pG(3′-5)p]; cyclic[C(2′-5′)pU(3′-5)p]; or cyclic[U(2′-5′)pC(3′-5′)p].

In certain embodiments, the cyclic-di-nucleotide has the following formula (cyclic[G(2′5′)pA(3′5′)p]):

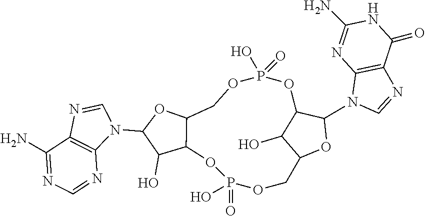 [SMILES: Nc1nc2c(ncn2C2OC3COP(=O)(O)OC4C(COP(=O)(O)OC2C3O)OC(n2cnc3c(N)ncnc32)C4O)c(=O)[nH]1]

or a salt thereof.

In certain embodiments, the cyclic-di-nucleotide has the following formula (cyclic[G(3′5′)pA(2′5′)p]):

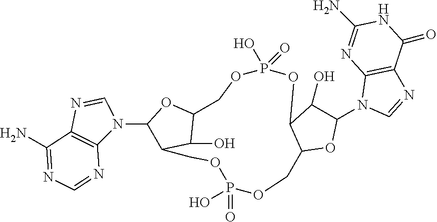 [SMILES: Nc1nc2c(ncn2C2OC3COP(=O)(O)OCC4C(O)C(COP(=O)(O)OC3C2O)OC4n2cnc3c(N)ncnc32)c(=O)[nH]1]

or a salt thereof.

In other embodiments, the cyclic-di-nucleotide has the following formula cyclic[G(2′5′)pA(2′5′)p]:

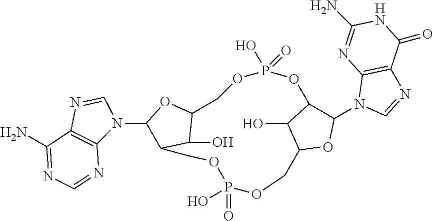 [SMILES: Nc1nc2c(ncn2C2OC3COP(=O)(O)OCC4C(O)C(COP(=O)(O)OC2C3O)OC4n2cnc3c(N)ncnc32)c(=O)[nH]1]

or a salt thereof.

In other embodiments, the cyclic-di-nucleotide has the following formula cyclic[A(2′5′)pA(3′5′)p]:

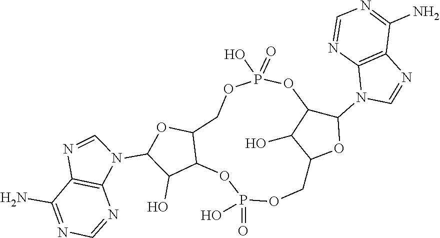 [SMILES: Nc1ncnc2c1ncn2C1OC2COP(=O)(O)OC3C(O)C(COP(=O)(O)OC2C1O)OC3n1cnc2c(N)ncnc21]

or a salt thereof.

In yet other embodiments, the cyclic-di-nucleotide has the following formula cyclic[G(2′5′)pG(3′5′)p]:

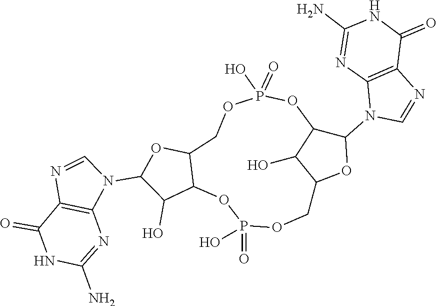 [SMILES: Nc1nc2c(ncn2C2OC3COP(=O)(O)OC4C(O)C(COP(=O)(O)OC3C2O)OC4n2cnc3c(=O)[nH]c(N)nc32)c(=O)[nH]1]

or a salt thereof.

In certain embodiments, the cyclic-di-nucleotide has the following formula cyclic[A(2′5′)pA(2′5′)p]:

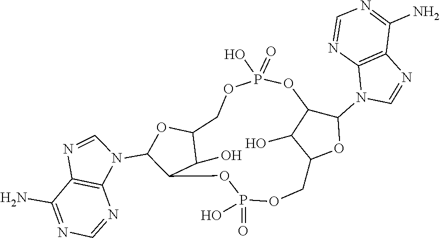 [SMILES: Nc1ncnc2c1ncn2C1OC2COP(=O)(O)OC3C(O)C(COP(=O)(O)OC1C2O)OC3n1cnc2c(N)ncnc21]

or a salt thereof.

In certain embodiments, the cyclic-di-nucleotide has the following formula cyclic[G(2′5′)pG(2′5′)p]:

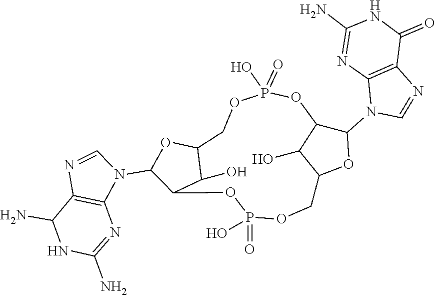 [SMILES: NC1=Nc2c(ncn2C2OC3COP(=O)(O)OC4C(O)C(COP(=O)(O)OC2C3O)OC4n2cnc3c(=O)[nH]c(N)nc32)C(N)N1]

or a salt thereof.

In certain embodiments, the cyclic-di-nucleotide has one of the following formulae:

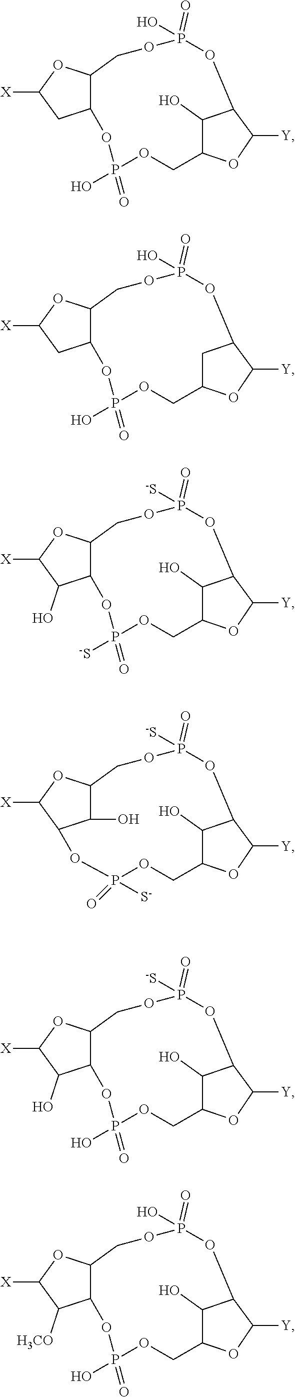

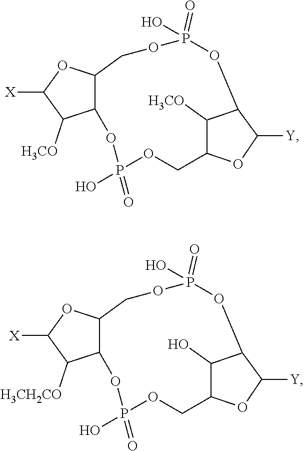

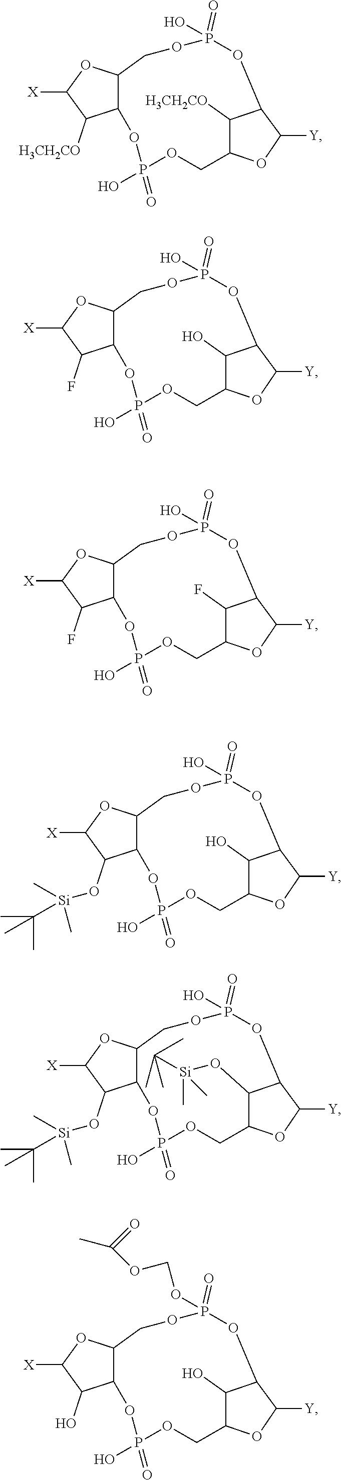

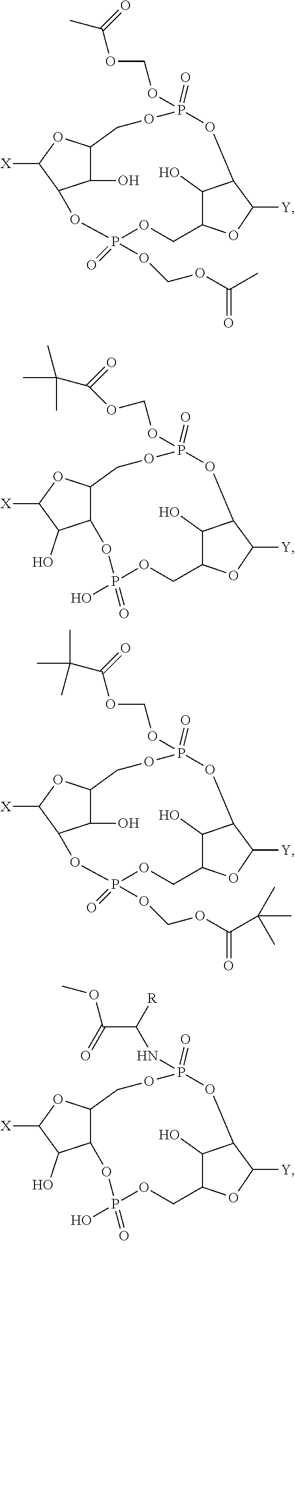

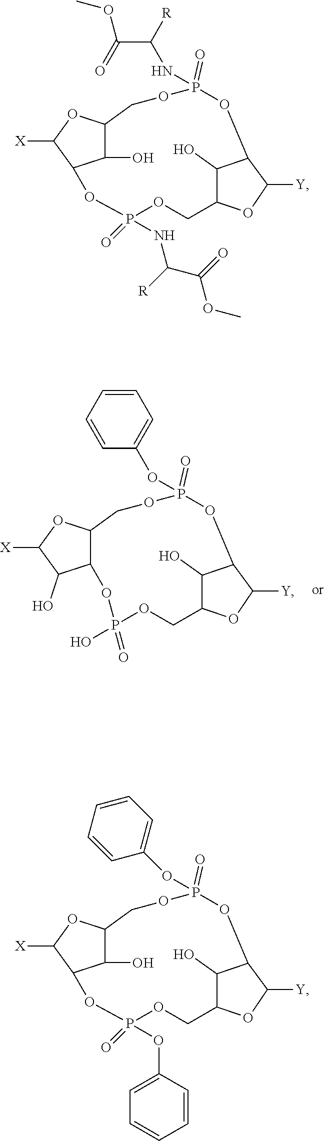

wherein R is any amino acid side chain, and X and Y are as defined above for formula (I)-(II), or a salt thereof.

CDN's of interest include, but are not limited to, those described by Dubensky et al. in U.S. Publication No. 2015/0056224. In certain embodiments, the CDN has the structure:

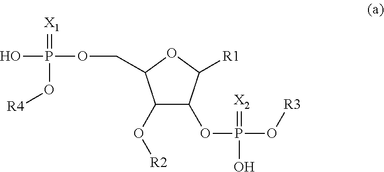

covalently linked to

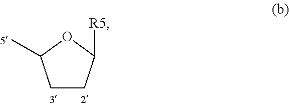

wherein:

R3 is a covalent bond to the 5′ carbon of (b),

R4 is a covalent bond to the 2′ or 3′ carbon of (b),

R1 is a purine linked through its N9 nitrogen to the ribose ring of (a),

R5 is a purine linked through its N9 nitrogen to the ribose ring of (b),

each of X₁ and X₂ are independently 0 or S,

R2 is H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, —OH, ═O, —COOR′ where R′ is H or lower alkyl, —CH₂OH, and —CONH₂, and the 2′ or 3′ carbon of (b) which is not in a covalent bond with (a) is —O—R6, wherein R6 is H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆alkoxy, —NO₂, —NH₂, —OH, ═O, —COOR′ where R′ is H or lower alkyl, —CH₂OH, and —CONH₂, or a prodrug or pharmaceutically acceptable salt thereof.

In certain cases, the CDN has one of the following formula: c-[G(2′,5′)pG(3′,5′)p], c-[A(2′,5′)pA(3′,5′)p], c-[G(2′,5′)pA(3′,5′)p] or c-[G(2′,5′)pA(3′,5′)p] where each p refers to a phosphate, thiophosphate or dithiophosphate internucleotide linkage. In certain instances, the CDN is a compound of the formula:

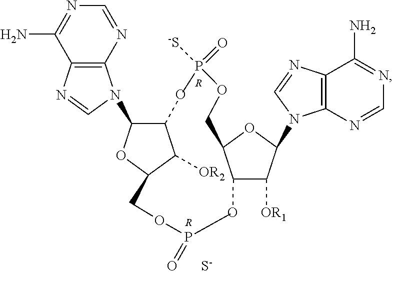

where R1 and R2 are each H, or a pharmaceutically acceptable salt thereof.

In certain cases, the CDN is a bisphosphorothioate analog of a naturally occurring CDN such as c-di-AMP. In some cases, the CDN is ADU-S100 or 2′3′-c-di-AM(PS)₂(Rp,Rp), also known as dithio-(Rp, Rp)-[cyclic[A(2′,5′)pA(3′,5′)p]] or (ML RR-S2 CDA), as described by Corrales et al. (“Direct Activation of STING in the Tumor Microenvironment Leads to Potent and Systemic Tumor Regression and Immunity” Cell Reports 11, 1018-1030, May 19, 2015).

### Methods

As summarized above, this disclosure provides methods of modulating the activity of a CDN transporter in order to modulate cell uptake of a CDN of interest. By increasing or decreasing the cell uptake of a CDN of interest, the activity of the CDN in the cell can be modulated to provide for a desired biological effect. CDN transporters (e.g., as described herein) can be modulated by contacting the cell, in vitro or in vivo, with a CDN transporter-modulating agent (e.g., as described herein).

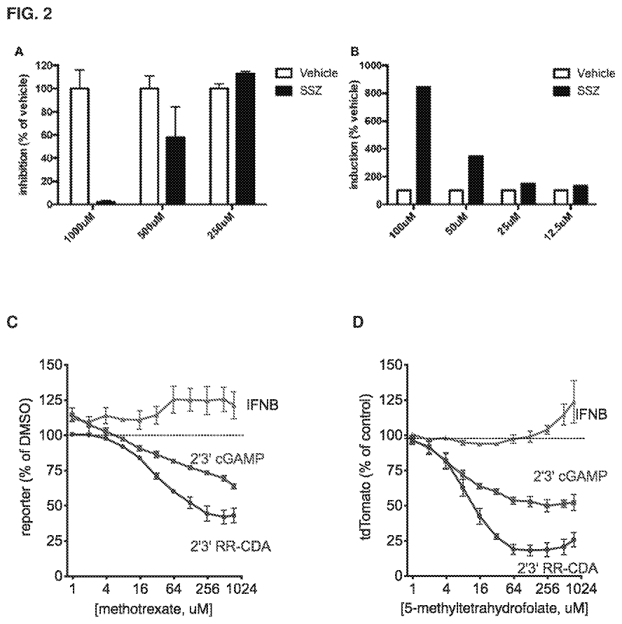

In some cases, the CDN transporter-modulating agent enhances or increases the activity or action of the CDN transporter. In some cases, the CDN transporter-modulating agent inhibits the activity or action of the CDN transporter. The response of the CDN transporter can be dependent on the amount of the CDN transporter-modulating agent that is utilized. For example, as shown in FIG. 2, panel A, Applicants demonstrated that a high concentration (e.g., 1000 μM or more) of sulfasalazine provided for complete blocking of the response to CDN stimulation in THP1 cells. In addition, low concentrations of sulfasalazine (e.g. 100 μM or less) significantly increased the responsiveness of cells to synthetic CDN compared to untreated cells (see e.g., FIG. 2, panel B). Based on Applicants' disclosure one of ordinary skill in the art could readily determine the amount of the CDN transporter-modulating agent that should be utilized to provide for a desired effect. As such, in some cases, agents that are capable of inhibiting a membrane transporter of interest at one concentration can be adapted for use in the subject methods to provide for enhancement of transporter activity.

In some embodiments, the cell is contacted with an amount of a CDN transporter-modulating agent effective to inhibit or decrease cellular uptake of a CDN. In some embodiments, the cell is contacted with an amount of a CDN transporter-modulating agent effective to increase or enhance cellular uptake of a CDN. In some cases, depending on the desired effect, the effective amount of the CDN transporter-modulating agent with which the cell is contacted ranges from 100 nM or more, such as 200 nM or more, 300 nM or more, 400 nM or more, 500 nM or more, 600 nM or more, 700 nM or more, 800 nM or more, 900 nM or more, 1 uM or more up to about 1 mM or more, such as up to about 3 mM, up to about 10 mM, up to about 30 mM or up to about 100 mM. In some cases, depending on the desired effect, the effective amount of the CDN transporter-modulating agent with which the cell is contacted is 100 nM or less, such as 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.3 nM or less, or even 0.1 nM or less.

In certain instances, the CDN transporter-modulating agent is a small molecule and the effective amount which is contacted with the cell or cellular sample, or administered to a subject, will generally contain between from about 1 mg to about 500 mg of the agent, in some cases, 25 mg or more, such as 50 mg or more, 100 mg or more, 200 mg or more, 300 mg or more, 400 mg or more, 500 mg or more, 600 mg or more, 800 mg or more, or 1000 mg or more.

In some embodiments, the subject methods include contacting a cell with an amount of the CDN transporter-modulating agent that is effective to inhibit transport of the CDN into the cell. In certain cases, the cellular uptake of a CDN into a cell is decreased by 10% or more, such as 20% or more, 30% or more, 40% or more, 50% or more, 60% or more, 70% or more, 80% or more, 90% or more, 100% or more, or even more relative to a control cell contacted with the CDN but not contacted with the CDN transporter-modulating agent.

In some aspects, the subject methods include contacting a cell with an amount of a CDN transporter-modulating agent that is effective to enhance transport of the CDN into the cell. In certain cases, the cellular uptake of a CDN into a cell is increased by 10% or more, such as 20% or more, 30% or more, 40% or more, 50% or more, 60% or more, 70% or more, 80% or more, 90% or more, 100% or more, or even more relative to a control cell contacted with the CDN but not contacted with the CDN transporter-modulating agent.

Any convenient method for determining cellular uptake can be utilized to assess and/or quantitate the effect of a CDN transporter-modulating agent. Methods of interest include, but are not limited to, those methods described by Rezgui et al. (“Precise quantification of cellular uptake of cell-penetrating peptides using fluorescence-activated cell sorting and fluorescence correlation spectroscopy”, Biochimica et Biophysica Acta (BBA)—Biomembranes, Volume 1858, Issue 7, Part A, July 2016, Pages 1499-1506) or bioanalytical methods such as liquid chromatography-mass spectrometry (LC-MS) to quantitate levels of CDNs such as described in Gao, et al., PNAS, 2015 (PMID 26371324).

Any convenient activities of CDNs can be targeted for modulation according to the subject methods in a variety of applications. In some cases, the CDN of interest provides an anti-tumor immune response via production of type I interferon (IFN) in a cell. As such, aspects of this disclosure include methods of increasing the production of a type I interferon (IFN) in a cell, e.g., in vitro or in vivo. By increasing type-I interferon production is meant that the subject methods increase type-I interferon production in a cell, as compared to a control, e.g., a cell that is not contacted with a CDN transporter modulating agent. The magnitude of the increase in type-I interferon production in a cell relative to what can be achieved with the CDN alone may vary, and in some instances is 10% or more, such as 20% or more, 30% or more, 40% or more, 50% or more, 60% or more, 70% or more, 80% or more, 90% or more, 100% or more, 2-fold or greater, 3-fold or greater, 4-fold or greater, 5-fold or greater, or 10-fold or greater, as compared to a suitable control. In those embodiments where, prior to practice of the subject methods, interferon production is not-detectable, the increase may result in detectable amounts of interferon production.

In some instances, the subject methods provide for increasing the production of a type I interferon (IFN)-stimulated gene in a cell, e.g., in vitro or in vivo. By increasing interferon-stimulated gene production is meant that the subject methods increase production of a interferon-stimulated gene or gene product in a cell, as compared to a control, e.g., a cell that is not contacted with a CDN transporter modulating agent. IFN-stimulated genes of interest include, but are not limited to, CXCL10, IRF7, IFIT3, ISG15 and RANTES. The magnitude of the increase in production in a cell relative to what can be achieved with the CDN alone may vary, and in some instances is 10% or more, such as 20% or more, 30% or more, 40% or more, 50% or more, 60% or more, 70% or more, 80% or more, 90% or more, 100% or more, 2-fold or greater, 3-fold or greater, 4-fold or greater, 5-fold or greater, or 10-fold or greater, as compared to a suitable control.

Type-I interferon production can be measured using any convenient method including, but not limited to, vesicular stomatitis virus (VSV) challenge bioassay, enzyme-linked immunosorbent assay (ELISA) replicon based bioassays or by using a reporter gene (e.g., luciferase) cloned under regulation of a type I interferon signaling pathway. See, e.g., Meager J. Immunol. Methods 261:21-36 (2002); Vrolijk et al. C. J. Virol. Methods 110:201-209 (2003); and Francois et al. Antimicrob Agents Chemother 49(9):3770-3775 (2005).

The methods may be used to increase the production of any convenient type I interferon including, but not limited to: IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). In some embodiments, the method is for increasing the production of IFN-α. In some embodiments, the method is for increasing the production of IFN-β.

In practicing embodiments of the methods provided herein, an effective amount of the CDN active agent (such as described above), is provided in the target cell or cells. As used herein “effective amount” or “efficacious amount” means the amount of the CDN that, when contacted with the cell, e.g., by being introduced into the cell in vitro, by being administered to a subject, etc., is sufficient to result in the desired outcome, e.g., increased levels of type I interferon in the cell. The “effective amount” will vary depending on cell and/or the organism and/or CDN and or the nature of the desired outcome and/or the disease and its severity and the age, weight, etc., of the subject to be treated.

An effective amount of CDN transporter-modulating agent is provided to the cells to result in a change in CDN levels in the cells. In some cases, an effective amount of CDN transporter-modulating agent is the amount to result in a 10% increase or more in the amount of CDN observed (directly or indirectly) in the cell, such as 20% or more, 30% or more, 40% or more, 50% or more, 60% or more, 70% or more, 80% or more, 90% or more, 100% or more, 2-fold or greater, 3-fold or greater, 4-fold or greater, 5-fold or greater, or 10-fold or greater, relative to a negative control, e.g., a cell not contacted with the CDN transporter-modulating agent. The amount of CDN observed may be measured by any suitable method, directly or indirectly. For example, the amount of type I interferon produced by the cell may be assessed after contact with the cyclic-di-nucleotide active agent(s), e.g., 2 hours, 4 hours, 8 hours, 12 hours, 24 hours, 36 hours, 48 hours, 72 hours or more after contact with the cyclic-di-nucleotide active agent(s).

Contact of the cell with the agent(s) may occur using any convenient protocol. The protocol may provide for in vitro or in vivo contact of the agent(s) with the target cell, depending on the location of the target cell. For example, where the target cell is an isolated cell, e.g., a cell in vitro (i.e., in culture), or a cell ex vivo (“ex vivo” being cells or organs are modified outside of the body, where such cells or organs are typically returned to a living body), the agent may be introduced directly to the cell under cell culture conditions permissive of viability of the target cell. The choice of method is generally dependent on the type of cell being contacted and the nature of the active agent, and the circumstances under which the transformation is taking place (e.g., in vitro, ex vivo, or in vivo). A general discussion of these methods can be found in Ausubel, et al, Short Protocols in Molecular Biology, 3rd ed., Wiley & Sons, 1995. As another example, where the target cell or cells are part of a multicellular organism, the active agent may be administered to the organism or subject in a manner such that the agent is able to contact the target cell(s), e.g., via an in vivo protocol. By “in vivo,” it is meant the agent is administered to a living body of an animal.

The CDN active agent(s) can be employed to increase the production of type I interferon in vivo. In these in vivo embodiments, the CDN and CDN transporter-modulating agents can be administered directly to the individual. In some embodiments, the CDN active agent administered to the subject contains a 2′-5′ phosphodiester linkage containing cyclic-di-nucleotide. The agent(s) may be administered by any suitable methods for the administration of peptides, small molecules or nucleic acids to a subject. The CDN and/or CDN transporter-modulating agents can be incorporated into a variety of formulations. More particularly, the agent(s) of the present disclosure can be formulated into pharmaceutical compositions by combination with appropriate pharmaceutically acceptable carriers or diluents. Pharmaceutical compositions that can be used in practicing the subject methods are described herein.

In some instances, an effective amount of the CDN agent is administered to the subject in conjunction with the CDN transporter-modulating agent. By an “effective amount” or a “therapeutically effective amount” of the agent it is meant an amount that is required to reduce the severity, the duration and/or the symptoms of the disease. In some embodiments, the effective amount of a pharmaceutical composition containing a CDN active agent for use in conjunction with the CDN transporter-modulating agent, as provided herein, is between 0.025 mg/kg and 1000 mg/kg body weight of a human subject. In certain embodiments, the pharmaceutical composition is administered to a human subject at an amount of 1000 mg/kg body weight or less, 950 mg/kg body weight or less, 900 mg/kg body weight or less, 850 mg/kg body weight or less, 800 mg/kg body weight or less, 750 mg/kg body weight or less, 700 mg/kg body weight or less, 650 mg/kg body weight or less, 600 mg/kg body weight or less, 550 mg/kg body weight or less, 500 mg/kg body weight or less, 450 mg/kg body weight or less, 400 mg/kg body weight or less, 350 mg/kg body weight or less, 300 mg/kg body weight or less, 250 mg/kg body weight or less, 200 mg/kg body weight or less, 150 mg/kg body weight or less, 100 mg/kg body weight or less, 95 mg/kg body weight or less, 90 mg/kg body weight or less, 85 mg/kg body weight or less, 80 mg/kg body weight or less, 75 mg/kg body weight or less, 70 mg/kg body weight or less, or 65 mg/kg body weight or less.

In some embodiments, the CDN is employed in mitotic or post-mitotic cells in vitro or ex vivo, i.e., to produce modified cells that can be reintroduced into an individual. Mitotic and post-mitotic cells of interest in these embodiments include any eukaryotic cell, e.g., pluripotent stem cells, for example, ES cells, iPS cells, and embryonic germ cells; somatic cells, for example, hematopoietic cells, fibroblasts, neurons, muscle cells, bone cells, vascular endothelial cells, gut cells, and the like, and their lineage-restricted progenitors and precursors; and neoplastic, or cancer, cells, i.e., cells demonstrating one or more properties associated with cancer cells, e.g., hyperproliferation, contact inhibition, the ability to invade other tissue, etc. In certain embodiments, the eukaryotic cells are cancer cells. In certain embodiments, the eukaryotic cells are hematopoietic cells, e.g., macrophages, NK cells, etc. Cells may be from any mammalian species, e.g., murine, rodent, canine, feline, equine, bovine, ovine, primate, human, etc. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. In some cases, the primary cell lines are maintained for fewer than 10 passages in vitro.

If the cells are primary cells, they may be harvested from an individual by any convenient method. For example, blood cells, e.g., leukocytes, e.g., macrophages, may be harvested by apheresis, leukocytapheresis, density gradient separation, etc., while cells from tissues such as skin, muscle, bone marrow, spleen, liver, pancreas, lung, intestine, stomach, etc. may be harvested by biopsy. An appropriate solution may be used for dispersion or suspension of the harvested cells. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells may be frozen in 10% DMSO, 50% serum, 40% buffered medium, or some other such solution as is commonly used in the art to preserve cells at such freezing temperatures, and thawed in a manner as commonly known in the art for thawing frozen cultured cells. The CDN active agent(s) may be produced by eukaryotic cells or by prokaryotic cells, it may be further processed by unfolding, e.g., heat denaturation, DTT reduction, etc. and may be further refolded, using methods known in the art.

### Combination Therapy

For use in the subject methods, the CDN active agent described herein may be administered in combination with the CDN transporter-modulating agent (e.g., as described herein). “In combination with” refers to uses where, for example, the first compound (e.g., CDN active agent) is administered during the entire course of administration of the second compound (e.g., CDN transporter-modulating agent); where the first compound is administered for a period of time that is overlapping with the administration of the second compound, e.g., where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds that may be administered in the same or different formulations, by the same of different routes, and in the same or different dosage form type.

Any convenient additional active agents, e.g., agents that find use in a combination therapeutic application with a CDN of interest, can also be utilized in conjunction with the CDN and CDN transporter-modulating agent in the subject methods. In some cases, the additional active agent is a chemotherapeutic agent or other cancer therapy, or an antiviral agent. In some embodiments, the additional cancer therapy comprises radiation therapy, surgery, chemotherapy, or an immunotherapy (for example, without limitation, an immunomodulator, an immune checkpoint inhibitor, a cellular immunotherapy, or a cancer vaccine). In some embodiments, the one or more additional cancer therapies comprise an inactivated tumor cell that expresses and secretes one or more cytokines or one or more heat shock proteins. In some embodiments, the cytokine is selected from the group consisting of GM-CSF, CCL20, CCL3, IL-12p70, and FLT-3 ligand. In some embodiments the heat shock protein is a gp96-Ig protein. In some cases, the additional active agent is an immune checkpoint inhibitor (e.g., CTLA-4, PD-1, TIM-3, Vista, BTLA, LAG-3, KIR, or TIGIT pathway antagonists, including, without limitation, PD-1 pathway blocking agents such as anti-PD-1 antibodies PDR001, nivolumab, pembrolizumab, SHR-1210, REGN2810 (cemiplimab), or pidilizumab, or PD-1 inhibitor AMP-224; PD-L1 inhibitors such as anti-PD-L1 antibodies BMS-936559, MPDL3280A(atezolizumab), MED14736 (durvalumab), or avelumab; anti-CTLA-4 antibodies such as ipilimumab, tremelimumab, 1131310, and AGEN1884; Vista inhibitors including anti-Vista antibodies; B7-H3 inhibitors including anti-B7-H3 antibodies; and CD70 inhibitors including anti-CD70 antibodies); Co-stimulatory checkpoint receptor agonist (e.g., CD40 agonists, including an anti-CD40 antibody; CD137 agonists, including an anti-CD137 antibody; GITR agonists, including an anti-GITR antibody; OX40 agonists, including an anti-OX40 antibody); an immune activating cytokine (e.g. IL-2, IL-12, IL-15); a TLR agonist (e.g., CpG or monophosphoryl lipid A); a RIG-1 agonist (e.g. 5′pp-dsRNA or 3p-hpRNA); a vaccine selected to stimulate an immune response to one or more cancer antigens, for example an inactivated or attenuated bacteria which induce innate immunity and is engineered to express cancer antigens (e.g., inactivated or attenuated Listeria monocytogenes); a therapeutic antibody that induces antibody-dependent cellular cytotoxicity; an immunomodulatory cell line; an antigen selected for the purpose of inducing an immune response, an agent which mediate innate immune activation (i) via Toll-like Receptors (TLRs) including, without limitation, TLR agonist (e.g., CpG or monophosphoryl lipid A), (ii) via (NOD)-like receptors (NLRs), (iii) via Retinoic acid inducible gene-based (RIG)-1-like receptors (RLRs), (iv) via C-type lectin receptors (CLRs), or (v) via pathogen-associated molecular patterns (“PAMPs”); a chemotherapeutic agent, etc.

In some embodiments, the immune checkpoint inhibitor is selected from the group consisting of a CTLA-4 pathway antagonist, a PD-1 pathway antagonist, a TIM-3 pathway antagonist, a Vista pathway antagonist, a BTLA pathway antagonist, a LAG-3 pathway antagonist, and a TIGIT pathway antagonist. In some embodiments, the immune checkpoint inhibitor is an anti-PD-1 antibody, an anti-PD-L1 antibody, an anti-CTLA-4 antibody, an anti-TIM-3 antibody, an anti-Vista antibody, an anti-BTLA antibody, an anti-B7-H3 antibody, an anti-CD70 antibody, an anti-KIR antibody or an anti-LAG-3 antibody. In some embodiments, the immune checkpoint inhibitor is selected from the group consisting of nivolumab, pembrolizumab, pidilizumab, cemiplimab, SHR-1210, PDR001, MED10680, AMP-224, ipilimumab, tremelimumab, 161310, AGEN1884, BMS-936559, atezolizumab, durvalumab, and avelumab.

Examples of chemotherapeutic agents for use in combination therapy include, but are not limited to, an indoleamine 2,3-dioxygenase (IDO1) inhibitor (e.g., epacadostat and navoximod), daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, lomustine (CCNU), carmustine, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, methylcyclohexylnitrosurea, nitrogen mustards (e.g. mechlorethamine, melphalan, cyclophosphamide, chlorambucil, uramustine, ifosfamide, bendamustine), prednimustine, estramustine phosphate, 6-mercaptopurine, 6-thioguanine, cytarabine, 5-azacytidine, hydroxyurea, deoxycoformycin, 4-hydroxyperoxycyclophosphor-amide, 5-fluorouracil (5-FU), 5-fluorodeoxyuridine (5-FUdR), methotrexate (MTX), colchicine, taxanes (e.g., taxol, docetaxel, cabazitaxel, larotaxel), vincristine, vinblastine, anhydrovinblastine, etoposide (VP-16), trimetrexate, irinotecan, topotecan, gemcitabine, decitabine, teniposide, cisplatin, carboplatin, diethylstilbestrol (DES), abiraterone acetate, altretamine, auristatin, bexarotene, bicalutamide, cachectin, cemadotin, cryptophycin, dolastatin, abemaciclib, acalabrutinib, afatinib, alectinib, axitinib, binimetinib, bosutinib, brigatinib, cabozantinib, ceritinib, cobimetinib, copanlisib, crizotinib, dabrafenib, dasatinib, encorafenib, entrectinib, erdafitinib, erlotinib, everolimus, gefitinib, ibrutinib, imatinib, lapatinib, lenvatinib, midostaurin, neratinib, nilotinib, osimertinib, palbociclib, pazopanib, ponatinib, regorafenib, ribociclib, ruxolitinib, sorafenib, sunitinib, temsirolimus, trametinib, vandetanib, vatalanib, vemurafenib, finasteride, flutamide, enasidenib, lenalidomide, liarozole, lonidamine, niraparib, olaparib, enzalutamide, mivobulin isethionate, abexinostat, belinostat, chidamide, entinostat, givinostat, panobinostat, quisinostat, resminostat, romidepsin, vorinostat, rhizoxin, rucaparib, sertenef, streptozocin, nilutamide, onapristone, sotrastaurin, tasonermin, tretinoin, venetoclax, vindesine sulfate, vinflunine, 3′,4′-didehydro-4′-deoxy-8′-norvin-caleukoblastine, BMS 184476, 2,3,4,5,6-pentafluoro-N-(3-fluoro-4-methoxyphenyl)benzene sulfonamide, and N,N-dimethyl-L-valyl-L-valyl-N-methyl-L-valyl-L-proly-1-Lproline-t-butylamide. Examples of additional agents for use in combination therapy of neoplastic disease include, but are not limited to, thalidomide, marimastat, COL-3, BMS-275291, squalamine, 2-ME, SU6668, neovastat, Medi-522, EMD121974, CAI, celecoxib, interleukin-12, IM862, TNP470, avastin, gleevec, herceptin, and mixtures thereof.

Examples of TLR agonists for use in combination therapy include, but are not limited to, Pam2Cys, Pam3Cys, Complete Freund's Adjuvant (CFA), monocyte activating lipopeptide-2 (MALP2), lipopeptide derived from Mycoplasma salivarium (FSL-1), Haemophilus influenzae type b outer membrane protein complex (Hib-OMPC), Poly I:C, Poly AU, Hiltonol® (poly-ICLC), monophosphoryl lipid A, lipopolysaccharide (LPS), bacterial flagellin, sialyl-Tn, imiquimod, resiquimod, lefitolimod, tilsotolimod, loxoribine, and CpG oligodeoxynucleotides (e.g., agatolimod, and unmethylated CpG dinucleotide).

Additional antiviral agents can also be delivered in conjunction with a CDN of interest in the treatment methods of this disclosure. For example, compounds that inhibit inosine monophosphate dehydrogenase (IMPDH) may have the potential to exert direct antiviral activity, and such compounds can be administered in a combination therapy, as described herein. Drugs that are effective inhibitors of hepatitis C NS3 protease may be administered in combination with the CDN, as described herein. Hepatitis C NS3 protease inhibitors inhibit viral replication. Other agents such as inhibitors of HCV NS3 helicase are also attractive drugs for combinational therapy and are contemplated for use in combination therapies described herein. Ribozymes such as Heptazyme™ and phosphorothioate oligonucleotides which are complementary to HCV protein sequences and which inhibit the expression of viral core proteins are also suitable for use in combination therapies described herein.

Examples of additional agents for use in combination therapy of multiple sclerosis include, but are not limited to; glatiramer; corticosteroids; muscle relaxants, such as Tizanidine (Zanaflex) and baclofen (Lioresal); medications to reduce fatigue, such as amantadine (Symmetrel) or modafinil (Provigil); and other medications that may also be used for depression, pain and bladder or bowel control problems that can be associated with MS.

Because of the adjuvant properties of the CDNs of the present disclosure, their use in the subject methods may also combined with other therapeutic modalities including other vaccines, adjuvants, antigen, antibodies, and immune modulators.

Examples of antibodies for use in combination therapy include, but are not limited to, muromonab-CD3, infliximab, omalizumab, daclizumab, rituximab, ibritumomab, tositumomab, cetuximab, trastuzumab, brentuximab vedotin, alemtuzumab, vitaxin, bevacizumab, and abciximab.

In the context of a combination therapy, combination therapy additional agents may be administered by the same route of administration (e.g., intrapulmonary, oral, enteral, etc.) that the CDN active agents are administered. In the alternative, the additional agents for use in combination therapy with the cyclic-di-nucleotide active agent may be administered by a different route of administration. Methods for co-administration with an additional therapeutic agent are well known in the art (Hardman, et al. (eds.) (2001) Goodman and Oilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

### Pharmaceutical Compositions

This disclosure provides a pharmaceutical composition that contains any of the CDN-transporter modulating agents (e.g., as described herein) and/or any of the CDN active agents (e.g., as described herein) and a pharmaceutically acceptable carrier. The pharmaceutical composition can include a CDN-transporter modulating agent as the only active agent. The pharmaceutical composition can include both a CDN-transporter modulating agent and a CDN. The subject pharmaceutical compositions find use in the kits and methods described herein.

The term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the agent of interest is administered. Such pharmaceutical carriers can be, for example, sterile liquids, such as dimethyl sulfoxide (DMSO) or saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The inhibitors can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin, hereby incorporated by reference herein in its entirety. Such compositions will contain a therapeutically effective amount of the mitochondrial transport protein (e.g., a Miro protein, a TRAK protein, or Khc) inhibitor, preferably in purified form, together with a suitable amount of carrier so as to provide the form for proper administration to the patient. The formulation should suit the mode of administration.

The pharmaceutical composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides or proteins, ions (e.g., sodium, potassium, calcium, magnesium, manganese) and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use may be sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The pharmaceutical composition can be formulated for intravenous, oral, via implant, transmucosal, transdermal, intramuscular, intrathecal, or subcutaneous administration. In some cases, the pharmaceutical composition is formulated for intravenous administration. In other cases, the pharmaceutical composition is formulated for subcutaneous administration. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGAs). Implantable systems include rods and discs, and can contain excipients such as PLGA and polycaprylactone. Osteopontin or nucleic acids of the invention can also be administered attached to particles using a gene gun.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

The pharmaceutical composition containing an active agent can be formulated to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. A BBB disrupting agent can be co-administered with the therapeutic compositions when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of the pharmaceutical composition behind the BBB may be by local delivery, for example by intrathecal delivery, e.g., through an Ommaya reservoir (see, e.g., U.S. Pat. Nos. 5,222,982 and 5,385,582, incorporated herein by reference); by bolus injection, e.g., by a syringe, e.g., intravitreally or intracranially; by continuous infusion, e.g., by cannulation, e.g., with convection (see, e.g., US Application No. 20070254842, incorporated here by reference); or by implanting a device upon which the inhibitor pharmaceutical composition has been reversibly affixed (see e.g., US Application Nos. 20080081064 and 20090196903, incorporated herein by reference).

In certain embodiments, the pharmaceutical composition containing the active agent is formulated in a delivery vehicle, e.g., to enhance passive cytosolic transport. Any convenient protocol may be employed to facilitate delivery of the CDN active agent across the plasma membrane of a cell and into the cytosol.

In some instances, the CDN and/or CDN transporter modulating active agent may be encapsulated in a delivery vehicle comprising liposomes in the pharmaceutical composition. Methods of using liposomes for drug delivery and other therapeutic uses are known in the art. See, e.g., U.S. Pat. Nos. 8,329,213, 6,465,008, 5,013,556, US Application No. 20070110798, and Andrews et al., Mol Pharm 2012 9:1118, which are incorporated herein by reference. Liposomes may be modified to render their surface more hydrophilic by adding polyethylene glycol (“pegylated”) to the bilayer, which increases their circulation time in the bloodstream. These are known as “stealth” liposomes and are especially useful as carriers for hydrophilic (water soluble) molecules.

In certain embodiments, nano- or microparticles made from biodegradable materials such as poly(lactic acid), poly(γ-glutamic acid), poly(glycolic acid), polylactic-co-glycolic acid, polyethylenimine, or alginate microparticles, and cationic microparticles, including dedrimers, such as cyclodextrins, may be employed as delivery vehicles for the active agents to promote cellular uptake. See, e.g., U.S. Pat. No. 8,187,571, Krishnamachari et al., Adv Drug Deliv Rev 2009 61:205, Garzon et al., 2005 Vaccine 23:1384, incorporated herein by reference.

In certain embodiments, the delivery vehicle for delivering the active agents can also be targeting delivery vehicles, e.g., a liposome containing one or more targeting moieties or biodistribution modifiers on the surface of the liposome. A targeting moiety can be any agent that is capable of specifically binding or interacting with a desired target. The specific binding agent can be any molecule that specifically binds to a protein, peptide, biomacromolecule, cell, tissue, etc. that is being targeted (e.g., protein, peptide, biomacromolecule, cell, tissue, etc. wherein the active agent exerts its desired effect). Depending on the nature of the target site, the specific binding agent can be, but is not limited to, an antibody against an epitope of a peptidic analyte, or any recognition molecule, such as a member of a specific binding pair. For example, suitable specific binding pairs include, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a peptide aptamer binding pair; and the like.

In certain embodiments, the specific binding moiety includes an antibody. In some cases, the specific binding moiety is a fragment of an antibody which retains specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The specific binding moiety may also include Fab′, Fv, F(ab′)2, and or other antibody fragments that retain specific binding to antigen.

In certain embodiments, the targeting moiety is a binding agent that specifically interacts with a molecule expressed on a tumor cell or an immune cell (e.g., CD4, CD8, CD69, CD62L, and the like), such that the targeting delivery vehicle containing the cyclic-di-nucleotide or STING active agents is delivered to the site of a tumor or to specific immune cells.

Where desired, any combinations of the above listed delivery vehicles may be used advantageously to enhance delivery of the active agents to the target cells.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection, an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, the pharmaceutical composition is supplied as a water free concentrate. In some embodiments, the pharmaceutical composition is supplied as a dry sterile lyophilized powder at a unit dosage of at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, or at least 75 mg.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In some embodiments, the pharmaceutical composition is formulated as a salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In certain embodiments, the pharmaceutical composition contains a prodrug derivative of any of the CDN active agents provided herein. Such prodrugs can be subsequently converted to an active form of the CDN in the body of the subject administered the pharmaceutical composition.

### Screening Assays

Aspects of the invention also include screening assays configured to identify CDN transporters. Screening assays of interest include methods of identifying transporters that modulate the activity of a CDN. In embodiments of such screening assays, cells expressing an interferon responsive reporter system are transduced with candidate transporter expression suppressors, e.g., guide RNAs, and then contacted with the CDN. Those cells hypo-responsive with respect to the CDN may be identified as having transporters that mediate CDN transport in to the cell. A variety of different types of cells may be screened in this manner to identify CDN transporters, where such cells include, but are not limited to: leukocytes, e.g., monocytes, macrophages, dendritic cells, etc. Any convenient reporter system may be present in the cell, where such reporter systems include those that produce a detectable protein label, e.g., fluorescent protein, enzyme, etc. Examples of fluorescent proteins of interest that may be employed as signal producing system labels include, but are not limited to, reef coral fluorescent proteins, e.g., as available under the Living Colors trademark from Takara BIO USA, and the like. Examples of enzymatic labels of interest include, but are not limited to: luciferase, SEAP, horse radish peroxidase, (3-galactosidase, etc. A specific screening assay and reagents employed therein is provided in the Experimental Section, below. As such, aspects of the invention further include screening assays designed to find CDN transporters, where the identification of transporters may find use in the identification of additional CDN transport modulatory agents, e.g., for us in methods described herein.

### Kits

Kits including a CDN transporter-modulating agent (e.g., as described herein) and a CDN active agent (e.g., as described herein) are provided. In some cases, the kit includes a CDN transporter-modulating agent to provide for enhanced cellular uptake of a CDN. In some cases, the kit includes a subject CDN transporter-modulating agent to provide for inhibition of cellular uptake of a CDN. In some cases, the kit includes a unit dose of the subject CDN active agents e.g., in an oral or injectable dose.

In the subject kits, the one or more components are present in the same or different containers, as may be convenient or desirable. In addition to the containers containing the components of the kits (e.g., unit doses) instructions can be included describing the use and attendant benefits of the CDN and CDN transporter-modulating agent in treating a pathological condition of interest. Instructions may be provided in a variety of different formats. In certain embodiments, the instructions may include complete protocols for practicing the subject methods or means for obtaining the same (e.g., a website URL directing the user to a webpage which provides the instructions), where these instructions may be printed on a substrate, where substrate may be one or more of: a package insert, the packaging, reagent containers and the like.

### Utility

This disclosure provides methods, compositions and kits that find use in a variety of applications. The subject methods find use in a variety of applications where it is desirable to either inhibit or enhance the cellular uptake of a CDN of interest in a target cell. Therapeutic applications of interest include, but are not limited to, cancer immunotherapy, antiviral applications, treatment of autoimmune or inflammatory disease, applications involving drug molecules that block or enhance CDN transporters (e.g., SLC19A transporters such as SLC19A1 or SLC46 transporters such as SLC46A1 or SLC46A3), and other in applications similar to those described herein. In some cases, this disclosure provides for methods and applications involving modulating (e.g., enhancing) transport of CDNs by membrane transporters into immune cells in vivo, leading to greater activation of the immune response.

Specific applications of interest include those in which a subject is treated for a disease condition that would benefit from an increase in type I interferon by providing the subject with a therapeutically effective amount of a CDN active agent. In some instances, it may be desirable to increase a type I interferon or STING mediated response in a healthy individual, e.g., for the prevention of a disease or condition. The subject methods can be applied to enhance uptake of administered CDNs by host cells for improved cancer immunotherapy. The subject methods can also be applied to enhance uptake of endogenously produced CDNs for improved cancer immunotherapy. In anti-cancer therapy, the subject methods can also be applied locally or systemically to inhibit or block uptake of the CDN active agent by target cells where it is desirable to alleviate the possible toxic effects of such a CDN, e.g., a CDN administered for anti-cancer treatment.

In some cases, gene expression profiles of tumor cells of a subject can be used to direct anti-cancer treatment with CDNs according to the subject methods. Aspects of the disclosure include a method of selecting a subject diagnosed with or suspected of having cancer who will benefit from treatment with a CDN. The gene expression profile of CDN transporters such as SLC19A1, SLC46A1 and/or SLC46A3 in the target cells can be utilized to identify patients for treatment according to the subject methods. In some instances, the method includes determining the gene expression profile of CDN transporters in cancer cells of a biological sample from a subject. Any convenient methods (e.g., as described herein) can be utilized to obtain a gene expression profile of genes of interest from the cancer cells. The gene expression profile from the cells of the subject can identify whether the subject has cancer cells susceptible to treatment with a CDN, e.g., because the cells express CDN transporters of interest at an elevated level, or the cells include an allele of a CDN transporter of interest that is capable of enhanced cell uptake of the CDN. In some cases, identifying the subject includes comparison of the subject's cells to a comparative cell standard that is representative of cancer cells from a plurality of individuals diagnosed with the same type of cancer the subject is diagnosed with or suspected of having. The cell standard can be a characterized cell line. Subjects who will benefit from treatment with a CDN are identified when the gene expression profile is determined to comprise a CDN transporter that modulates cell uptake of the CDN. A selected subject can be treated for cancer with a CDN according to the methods described herein.

In some embodiments, the evaluation protocol includes a genotyping assay. The term “genotype or genotyping” means the combination of alleles that determines a specific trait of an individual or the particular alleles at specified loci present in an organism. The genotyping assay may genotype one or more genes, where genes of interest include, but are not limited to: CDN transporters such as SLC19A1, SLC46A1 and/or SLC46A3. In some instances, the genotyping comprises assaying for the presence of a polymorphism. The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long. In some instances, the genotyping includes assessing a single nucleotide polymorphism (SNP), including assaying 2 or more SNPs, e.g., 3 or more SNPs. In some instances, the genotyping assay is a haplotyping assay. The term “haplotype” as used herein is intended to refer to a set of alleles that are inherited together as a group (are in linkage disequilibrium) at statistically significant levels (P_{corr}<0.05). Haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Amplification methods that may be employed include those selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the gene loci, either flanking the marker of interest (as required for PCR amplification) or directly overlapping the marker (as in ASO hybridization). In some embodiments, the sample is hybridized with a set of primers, which hybridize 5′ and 3′ in a sense or antisense sequence to the disease associated allele, and is subjected to a PCR amplification.

The present invention also provides a method of inhibiting type I interferon production mediated by the cGAS-STING pathway. In some embodiments, subjects suitable for treatment with a method described herein include individuals having an immunological or inflammatory disease or disorder including, but not limited to a cancer, an autoimmune disease or disorder, an allergic reaction, a chronic infectious disease and an immunodeficiency disease or disorder. The subject methods can be applied with the purpose of inhibiting CDN uptake and signaling in autoimmune/inflammatory diseases linked to aberrant CDN signaling, In some embodiments, the disease or disorder can be a type I interferonopathy (e.g., Aicardi-Goutieres Syndrome, Sjögren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes. In other embodiments, the disease or disorder can be an inflammatory disorder (e.g., atherosclerosis, dermatomyositis, SIRS, sepsis, septic shock, atherosclerosis, celiac disease, interstitial cystitis, transplant rejection, rheumatoid arthritis, juvenile rheumatoid arthritis, inflammatory bowel disease (ulcerative colitis, Crohn's disease), age-related macular degeneration, IgA nephropathy, glomerulonephritis, vasculitis, polymyositis, or Wegener's disease).

The subject methods can be applied is a variety of other applications, including any convenient applications where drugs that block or enhance CDN transporters, such as SLC19A1, SLC46A1, or SLC46A3 find use.

In some embodiments, subjects suitable for treatment with a method of the present invention include individuals having a cellular proliferative disease, such as a neoplastic disease (e.g., cancer). Cellular proliferative disease is characterized by the undesired propagation of cells, including, but not limited to, neoplastic disease conditions, e.g., cancer. Examples of cellular proliferative disease include, but are not limited to, abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying, for example, rheumatoid arthritis, psoriasis, diabetic retinopathy, other ocular angiogenic diseases such as retinopathy of prematurity (retrolental fibroplastic), macular degeneration, corneal graft rejection, neurovascular glaucoma and Oster Webber syndrome, psoriasis, restenosis, fungal, parasitic and viral infections such cytomegaloviral infections. Subjects to be treated according to the methods of the invention include any individual having any of the above-mentioned disorders.

The subject methods can be applied with the purpose of increasing intercellular 2′3′-cGAMP signaling between virus-infected and uninfected cells for amplification of anti-viral immunity. In some embodiments, subjects suitable for treatment with a subject method include individuals who have been clinically diagnosed as infected with a virus. In some embodiments, the virus is a hepatitis virus (e.g., HAV, HBV, HCV, delta, etc.), particularly HCV, are suitable for treatment with the methods of the instant invention. Individuals who are infected with HCV are identified as having HCV RNA in their blood, and/or having anti-HCV antibody in their serum. Such individuals include naïve individuals (e.g., individuals not previously treated for HCV, particularly those who have not previously received IFN-α-based or ribavirin-based therapy) and individuals who have failed prior treatment for HCV.

In certain embodiments, subjects suitable for treatment with a method of the present invention include individuals having multiple sclerosis. Multiple sclerosis refers to an autoimmune neurodegenerative disease, which is marked by inflammation within the central nervous system with lymphocyte attack against myelin produced by oligodendrocytes, plaque formation and demyelization with destruction of the myelin sheath of axons in the brain and spinal cord, leading to significant neurological disability over time. Typically, at onset an otherwise healthy person presents with the acute or sub-acute onset of neurological symptomatology (attack) manifested by unilateral loss of vision, vertigo, ataxia, dyscoordination, gait difficulties, sensory impairment characterized by paresthesia, dysesthesia, sensory loss, urinary disturbances until incontinence, diplopia, dysarthria or various degrees of motor weakness until paralysis. The symptoms may be painless, remain for several days to a few weeks, and then partially or completely resolve. After a period of remission, a second attack will occur. During this period after the first attack, the patient is defined to suffer from probable MS. Probable MS patients may remain undiagnosed for years. When the second attack occurs the diagnosis of clinically definite MS (CDMS) is made (Poser criteria 1983; C. M. Poser et al., Ann. Neurol. 1983; 13, 227).

The following examples are offered by way of illustration and not by way of limitation.

### Examples

Targeting the Transporter SLC19A1 and Other Transporters with Similar Activity in Patients with Cancer and Inflammatory Diseases.

CDNs are charged molecules that cannot readily pass through lipid bilayers, indicating that entry of a CDN to the cytosol of a cell is facilitated by a membrane transporter. Membrane-bound transporters can provide for the transport of endogenous substrates across a cell membrane, including substrates such as amino acids and oligopeptides, glucose and other sugars, inorganic cations and anions, bile salts, organic anions, acetyl coenzyme A, essential metals, biogenic amines, neurotransmitters, vitamins, fatty acids and lipids, nucleosides, ammonium, choline, thyroid hormone and urea. Whereas the membrane transporters exist for many endogenous substrates, certain drugs and other non-naturally occurring molecules are able to ‘hitch-hike’ on one or more of these transporters, to provide for entry (or exit) of the cell.

Applicants discovered that certain membrane transporters are involved in cellular uptake of CDNs and that these transporters can be targeted to modulate the transport of a CDN of interest into the cell. By increasing or decreasing the cellular uptake of a CDN of interest into a cell, the activity of the CDN in the cell can be modulated to provide for a desired biological effect.

The natural anti-tumor immune response is often weak. A much amplified anti-tumor immune response occurs when STING agonists, such as cyclic dinucleotides (CDNs), are injected into the tumor microenvironment, leading to powerful immune activation and tumor regression (Corrales et al. “Direct Activation of STING in the Tumor Microenvironment Leads to Potent and Systemic Tumor Regression and Immunity.” Cell Rep. 11, 1018-30 (2015)). Several STING agonists are promising candidates for cancer immunotherapy, including synthetic CDNs that are analogues of 2′3′-cGAMP (Corrales, L. & Gajewski, T. F. “Molecular Pathways: Targeting the Stimulator of Interferon Genes (STING) in the Immunotherapy of Cancer.” Clin. Cancer Res. 21, 4774-9 (2015)). The injection of CDNs into the tumor microenvironment induces biological responses in a wide variety of immune and non-immune cells (Corrales, Cell Rep. 11, 1018-30 (2015); Francica et al. “TNFα and Radioresistant Stromal Cells Are Essential for Therapeutic Efficacy of Cyclic Dinucleotide STING Agonists in Nonimmunogenic Tumors.” Cancer Immunol. Res. 6, 422-433 (2018)). However, the pathway by which extracellular CDNs enter cells and activate STING was unknown. CDNs are charged molecules that cannot readily pass through lipid bilayers, suggesting that entry to the cytosol is facilitated in some manner.

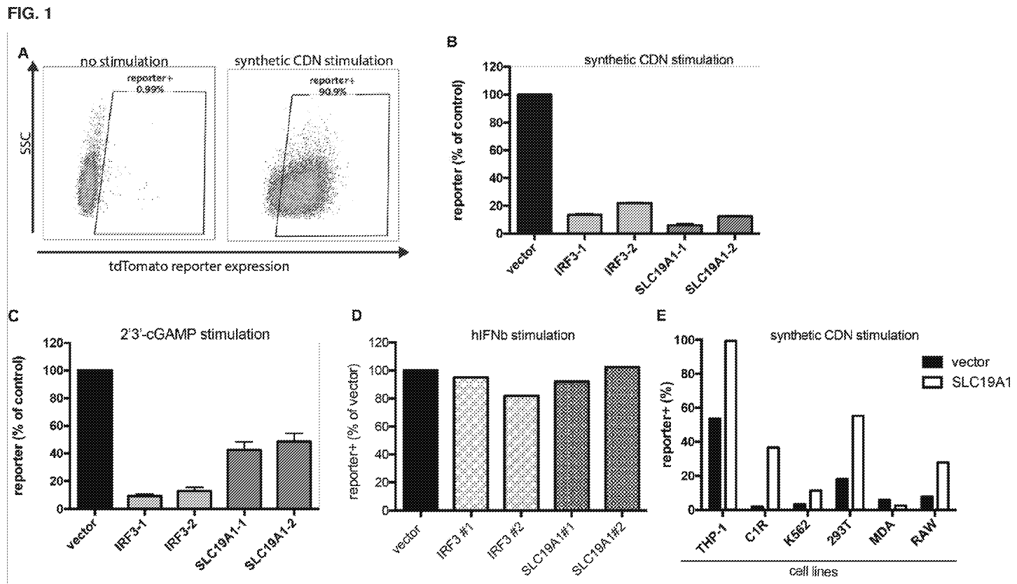

To identify genes encoding molecules involved in this process, a reporter system was created to detect STING activation in response to extracellular CDNs, in order to perform a genetic screen. The reporter was composed of an Interferon Stimulatory Response Element (ISRE) cassette driving the expression of the fluorescent protein tdTomato. The reporter system was expressed in the human monocytic cell line THP-1 to confirm that the tdTomato reporter was induced in response to stimulation with human interferon-beta (hIFNβ) or a synthetic CDN (FIG. 1A). Subsequently, THP-1 cells containing the reporter were transduced with a genome-wide library of guide RNAs (gRNAs), which suppress expression of target genes. The library targeted 20,000 genes within the human genome. The gRNA library-expressing THP-1 cells were stimulated with synthetic CDN, and the cells were subsequently sorted into two populations based on either low (hyporesponsive) or high (hyper-responsive) levels of tdTomato reporter expression. These populations were sequenced to reveal the RNA targets. As expected, gRNAs targeting components of the STING pathway, including IRF3 and STING itself, were enriched in the hyporesponsive cells and depleted in the hyper-responsive cells, corroborating the success of the screening procedure. Other “hits” in the screen were examined for their role in CDN transport.

One of the most significant hits in the hypo-responsive population was a gene encoding the cell surface transporter protein SLC19A1. This transporter is ubiquitously expressed and is known to transport folates and folate-derivatives into the cytosol of cells (Hou & Matherly, “Biology of the major facilitative folate transporters SLC19A1 and SLC46A1.” Current Topics in Membranes 73, (Elsevier Inc., 2014)). To test the role of SLC19A1 in the response to extracellular CDNs, THP-1 cell lines were created in which SLC19A1 was knocked-down or knocked-out, using conventional methods. SLC19A1 knockdown was confirmed and cells were stimulated with synthetic CDN or hIFNβ. Indeed, knocking down SLC19A1 expression significantly affected synthetic CDN stimulation (see FIG. 1, panel B), whereas no effect was seen upon hIFNβ stimulation (FIG. 1, panel D). Panel C shows that knocking down SLC19A1 also results in reduced responses to 2′3′ cGAMP. Furthermore, overexpression of SLC19A1 in THP-1 cells and other tumor cell lines increased their sensitivity to synthetic CDN (FIG. 1, panel E).

FIG. 1. Panel A) Flow cytometry-generated dot plots of THP-1 cells expressing an ISRE-driven tdTomato reporter. Reporter expression was induced upon stimulation with the synthetic CDN (1.7 μg/ml) for 20 h. Panel B) Validation of targets identified by a genome-wide screen using THP-1 cells described in (Panel A) using conventional methods. Cells expressed a control vector or IRF3 or SLC19A1 gRNA. Cells were stimulated with (Panel B) synthetic CDN (1.7 μg/ml) or (Panel D) human interferon-beta (hIFNβ) (100 ng/ml) for 20 h. Cells were analyzed for reporter by flow cytometry as in (A). E) Indicated cell lines expressing SLC19A1 or vector only were stimulated with synthetic CDN (1.7 μg/ml). Reporter expression was measured 20 h after stimulation.

The transport activity of folate by SLC19A1 can be altered by a variety of organic and inorganic anions, including the compound sulfasalazine (Jansen et al. “Sulfasalazine is a potent inhibitor of the reduced folate carrier: Implications for combination therapies with methotrexate in rheumatoid arthritis.” Arthritis Rheum. 50, 2130-2139 (2004); Goldman, “The characteristics of the membrane transport of amethopterin and the naturally occurring folates.” Ann. N. Y. Acad. Sci. 186, 400-22 (1971)). At high doses, sulfasalazine non-competitively inhibits the transport of folates and folate-derivatives by SLC19A1 (Jansen et al., Arthritis Rheum. 50, 2130-2139 (2004)). We therefore tested whether sulfasalazine blocks the response of cells to CDNs. A high concentration (1000 μM) of sulfasalazine almost completely blocked the response to synthetic CDN stimulation in THP1 cells (FIG. 2, panel A). Surprisingly, low concentrations of sulfasalazine (e.g. 100 μM) significantly increased the responsiveness of cells to synthetic CDN compared to untreated cells (FIG. 2, panel B). The response of the cells to hIFNβ was only marginally affected by either low or high sulfasalazine concentrations. These data suggest that sulfasalazine blocks or enhances CDN uptake, depending on the dose.

FIG. 2. Panel A) THP-1 cells were stimulated with synthetic CDN (2.5 μg/ml) in the presence of high concentrations of sulfasalazine (SSZ) or vehicle. Reporter expression was measured 20 h post stimulation. Panel B) THP-1 cells were stimulated with synthetic CDN (1.25 μg/ml) in the presence of low concentrations of SSZ or vehicle. Reporter expression was measured 20 h post stimulation.

Sulfasalazine is an approved treatment for the autoimmune/inflammatory diseases rheumatoid arthritis (RA) and inflammatory bowel disease (including ulcerative colitis and Crohn's disease). The mechanism of action is unknown. CDN signaling between cells may play a role in the initiation and/or persistence of RA, inflammatory bowel disease and possibly additional inflammatory disorders. Hence, drugs targeting SLC19A1 may be therapeutic in several inflammatory disorders.

THP-1 cells were incubated with increasing concentrations of the competitive inhibitors methotrexate (FIG. 2 panel C), 5-methyl tetrahydrofolate (5-methyl THF, FIG. 2 panel D) or DMSO as vehicle control, before stimulating with 2′3′-RR CDA (1.25 g/ml), 2′3′-cGAMP (15 g/ml) or hIFN- (100 ng/ml). After 18 h, tdTomato reporter expression was analyzed by flow cytometry. For each stimulant, the data were normalized to the DMSO controls.

The role of SLC19A1 in responses of cells to other CDNs, including the mammalian CDN 2′3′-cGAMP, and bacterial cyclic di-AMP (CDA) was also evaluated. Knocking down SLC19A1 expression reduced the response to CDA, to a similar extent as it did for the synthetic CDN. The response to mammalian 2′3′-cGAMP was also affected by SLC19A1, albeit to a lesser extent (up to 50% reduction compared to the control response). Several other transporters were identified in the screen. Knocking down at least one of these transporters, SLC46A3, modestly inhibited responses of the THP-1 cells to synthetic CDN. Hence other transporters may also participate in uptake of CDNs by cells.

Two other transporter molecules were also tested, including SLC46A3 and SLC46A1. SLC46A3 was identified in the screen. SCL46A1, like SLC19A1, is a major folate receptor so its activity was tested as well. Overexpression of SLC46A1 increased the sensitivity of THP-1 cells to 2′3′-cGAMP (10 μg/ml) or synthetic CDN (1.67 μg/ml) stimulation. Reducing the expression of SLC46A3 in THP-1 cells decreased the response to 2′3′-cGAMP and synthetic CDN. These data indicate that SLC46A1 and SLC46A3 are both transporters for 2′3′-cGAMP and synthetic CDN.

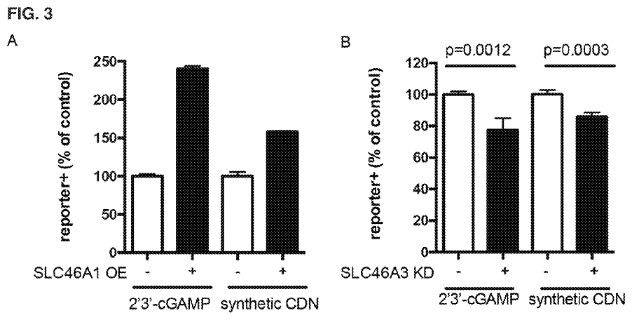

FIG. 3, panels A-B show that overexpression of SLC46A1 increases the responses of cells to CDNs (Panel A), and that decreasing the expression of SLC46A3 decreases the response (Panel B). Panel A: Control THP-1 cells (−) or THP-1 cells overexpressing (OE) SLC46A1 were stimulated with 2′3′-cGAMP (10 μg/ml) or synthetic CDN (1.67 μg/ml) and reporter expression was measured 22 h later by flow cytometry. Panel B: Control THP-1 cells (−) or THP-1 cells expressing gRNAs targeting SLC46A3 (knockdown; KD) were stimulated with 2′3′-cGAMP (10 μg/ml) or synthetic CDN (1.67 μg/ml) and reporter expression was measured 18 h later by flow cytometry.

SLC19A1 expression and activity was found to substantially affect stimulation by both synthetic and natural CDNs. Targeting this transporter and other transporters with related activity may thus alter the immune response induced by these critical proinflammatory molecules.

Notwithstanding the appended claims, the disclosure is also defined by the following clauses:

1. A method of modulating activity of a cyclic dinucleotide (CDN) in a cell, the method comprising:
contacting a cell with:

a CDN; and

an amount of a CDN transporter-modulating agent effective to modulate transport of the CDN into the cell;

to modulate activity of the CDN in the cell.
2. The method according to clause 1, wherein the CDN transporter-modulating agent modulates a transporter of folate or a folate derivative.
3. The method according to clause 1 or 2, wherein the CDN transporter-modulating agent modulates SLC19A1-mediated transport of the CDN into the cell.
4. The method according to clause 1 or 2, wherein the CDN transporter-modulating agent modulates SLC46A1-mediated transport of the CDN into the cell.
5. The method according to clause 1 or 2, wherein the CDN transporter-modulating agent modulates SLC46A3-mediated transport of the CDN into the cell.
6. The method according to any one of clauses 1-5, wherein contacting the cell with the amount of the CDN transporter-modulating agent is effective to inhibit transport of the CDN into the cell.
7. The method according to any one of clauses 1-5, wherein contacting the cell with the amount of the CDN transporter-modulating agent is effective to enhance transport of the CDN into the cell.
8. The method according to clause 7, wherein modulating the activity of the CDN comprises increasing production of type I interferon in the cell (e.g., STING-dependent type I interferon production).
9. The method according to clause 8, wherein the type I interferon is interferon alpha.
10. The method according to clause 8, wherein the type I interferon is interferon beta.
11. The method according to any one of clauses 1-10, wherein the cell is in vitro.
12. The method according to any one of clauses 1-10, wherein the cell is in vivo.
13. The method according to clause 12, wherein the contacting comprises administration of the CDN transporter-modulating agent to a subject.
14. The method according to clause 13, wherein the subject has a viral infection.
15. The method according to clause 13, wherein the subject has a bacterial infection.
16. The method according to clause 13, wherein the subject has neoplastic disease.
17. The method according to clause 16, wherein modulating the activity of the CDN comprises enhancing uptake of administered CDN by host cells for improved cancer immunotherapy of the subject.
18. The method according to clause 16, wherein modulating the activity of the CDN comprises inhibiting CDN uptake locally or systemically to alleviate a toxic effect of a CDN administered for anti-cancer treatment.
19. The method according to clause 14, wherein modulating the activity of the CDN comprises increasing intercellular CDN (e.g., 2′,3′-cGAMP) signaling between virus-infected and uninfected cells for amplification of anti-viral immunity.
20. The method according to clause 13, wherein modulating the activity of the CDN comprises inhibiting 2′,3′-cGAMP uptake and signaling in a subject having an autoimmune or inflammatory disease linked to aberrant 2′3′-cGAMP signaling.
21. The method according to clause 20, wherein the autoimmune or inflammatory disease is selected from Aicardi-Goutieres Syndrome, Sjögren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes and Type 2 diabetes.
22. The method according to any one of clauses 12-21, wherein the subject is mammal.
23. The method according to clause 22, wherein the mammal is a human.
24. The method according to any one of clauses 1-23, wherein the contacting comprises contacting the cell with composition comprising a CDN.
25. The method according to any one of clauses 1-23, wherein the contacting comprises contacting the cell with an agent that endogenously produces the CDN in the cell.
26. The method according to any one of clauses 1-25, wherein the CDN is a naturally occurring CDN.
27. The method according to any one of clauses 1-25, wherein the CDN is a non-naturally occurring or synthetic CDN.
28. The method according to any one of clauses 1-27, wherein the CDN comprises two 2′-5′ phosphodiester linkages.
29. The method according to any one of clauses 1-27, wherein the CDN comprises a 2′-5′ phosphodiester linkage and a 3′-5′ phosphodiester linkage.
30. The method according to clause 29, wherein the CDN has the formula:

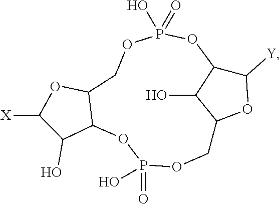

wherein X and Y are each independently:

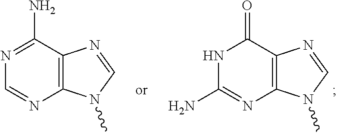

or a salt thereof.
31. The method according to clause 30, wherein the CDN is cyclic[G(2′5′)pG(3′5′)p].
32. The method according to clause 30, wherein the CDN is cyclic[A(2′5′)pA(3′5′)p].
33. The method according to clause 30, wherein the CDN is cyclic[G(2′5′)pA(3′5′)p].
34. The method according to clause 30, wherein the CDN is cyclic[A(2′5′)pG(3′5′)p].
35. The method according to any one of clauses 1-34, wherein the CDN transporter-modulating agent is selected from small molecule, organic anion, inorganic anion, an antibody or fragment thereof and an RNAi agent.
36. The method according to any one of clauses 1-35, wherein the CDN transporter-modulating agent is sulfasalazine or a salt thereof.
37. The method according to clause 35, wherein the CDN transporter-modulating agent is a SLC19A1-binding antibody or a fragment thereof.
38. The method according to clause 37, wherein the SLC19A1-binding antibody or a fragment thereof targets a first epitope of a SLC19A1 protein to inhibit SLC19A1-mediated transport of the CDN.
39. The method according to clause 37, wherein the SLC19A1-binding antibody or a fragment thereof targets a second epitope of a SLC19A1 protein to enhance SLC19A1-mediated transport of the CDN.
40. The method according to clause 35, wherein the CDN transporter-modulating agent is a SLC46A1-binding antibody or a fragment thereof.
41. The method according to clause 40, wherein the SLC46A1-binding antibody or a fragment thereof targets a first epitope of a SLC46A1 protein to inhibit SLC46A1-mediated transport of the CDN.
42. The method according to clause 40, wherein the SLC46A1-binding antibody or a fragment thereof targets a second epitope of a SLC46A1 protein to enhance SLC46A1-mediated transport of the CDN.
43. The method according to clause 35, wherein the CDN transporter-modulating agent is a SLC46A3-binding antibody or a fragment thereof.
44. The method according to clause 43, wherein the SLC46A3-binding antibody or a fragment thereof targets a first epitope of a SLC46A3 protein to inhibit SLC46A3-mediated transport of the CDN.
45. The method according to clause 43, wherein the SLC46A3-binding antibody or a fragment thereof targets a second epitope of a SLC46A3 protein to enhance SLC46A3-mediated transport of the CDN.
46. The method according to clause 35, wherein the CDN transporter-modulating agent is an RNAi agent that modulates expression of SLC19A1 in the cell.
47. The method according to clause 35, wherein the CDN transporter-modulating agent is an RNAi agent that modulates expression of SLC46A1 in the cell.
48. The method according to clause 35, wherein the CDN transporter-modulating agent is an RNAi agent that modulates expression of SLC46A3 in the cell.
49. A method of selecting a subject diagnosed with or suspected of having cancer who will benefit from treatment with a CDN, the method comprising:

determining the gene expression profile of, or genotyping one or more, CDN transporters in cancer cells of a biological sample from a subject;

selecting a subject who will benefit from treatment with a CDN when the gene expression profile or genotype is determined to comprise a CDN transporter that modulates cell uptake of the CDN; and

administering an effective amount of the CDN to the subject to treat the subject for cancer.

50. The method according to clause 49, wherein the CDN transporter that modulates cell uptake of the CDN is selected from SLC46A1, SLC46A1 and SLC46A3.
51. The method according to any one of clauses 49-50, wherein the gene expression profile is determined to comprise an allele of a CDN transporter capable of enhanced cell uptake of the CDN.
52. The method according to clause 50, wherein the gene expression profile is determined to comprise an elevated level of expression of SLC46A1, SLC46A1 and/or SLC46A3 relative to a cell standard.
53. The method according to clause 52, wherein the cell standard is representative of cancer cells from a plurality of individuals diagnosed with the same type of cancer the subject is diagnosed with or suspected of having.
54. The method according to clause 53, wherein the cell standard is a characterized cell line.
55. The method according to any one of clauses 49-54, wherein the cancer is a solid tumor cancer.
56. The method according to any one of clauses 49-53, further comprising administering to the subject an effective amount of an additional chemotherapeutic agent or cancer therapy.
57. The method according to any one of clauses 49-56, further comprising administering a CDN transporter-modulating agent to the subject.
58. A composition comprising:

a CDN; and

a CDN transporter-modulating agent.

59. The composition according to clause 58, wherein the CDN is naturally occurring.
60. The composition according to clause 58, wherein the CDN is a non-naturally occurring or synthetic CDN.
61. The composition according to any one of clauses 58-60, wherein the CDN comprises two 2′-5′ phosphodiester linkages.
62. The composition according to any one of clauses 58-60, wherein the CDN comprises two 3′-5′ phosphodiester linkages.
63. The composition according to any one of clauses 58-60, wherein the CDN comprises a 2′-5′ phosphodiester linkage and a 3′-5′ phosphodiester linkage.
64. The composition according to clause 63, wherein the CDN has the formula:

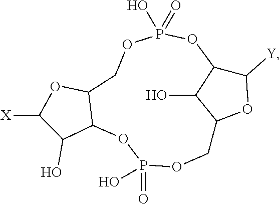

wherein X and Y are each independently:

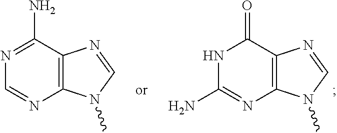

or a salt thereof.
65. The composition according to clause 64, wherein the CDN is cyclic[G(2′5′)pG(3′5′)p].
66. The composition according to clause 64, wherein the CDN is cyclic[A(2′5′)pA(3′5′)p].
67. The composition according to clause 64, wherein the CDN is cyclic[G(2′5′)pA(3′5′)p].
68. The composition according to clause 64, wherein the CDN is cyclic[A(2′5′)pG(3′5′)p].
69. The composition according to any one of clauses 58-68, wherein the CDN transporter-modulating agent modulates a transporter of folate or a folate derivative.
70. The composition according to any one of clauses 58-68, wherein the CDN transporter-modulating agent modulates SLC19A1.
71. The composition according to any one of clauses 58-68, wherein the CDN transporter-modulating agent modulates SLC46A3.
72. The composition according to any one of clauses 68-71, wherein, the CDN transporter-modulating agent is selected from small molecule, organic anion, inorganic anion, an antibody or fragment thereof and an RNAi agent.
73. The composition according to clause 72, wherein the CDN transporter-modulating agent is sulfasalazine or a salt thereof.
74. The composition according to clause 72, wherein the CDN transporter-modulating agent is a SLC19A1-binding antibody or a fragment thereof.
75. The composition according to clause 74, wherein the SLC19A1-binding antibody or a fragment thereof targets a first epitope of a SLC19A1 protein that inhibits SLC19A1-mediated transport of the CDN.
76. The composition according to clause 74, wherein the SLC19A1-binding antibody or a fragment thereof targets a second epitope of a SLC19A1 protein that enhances SLC19A1-mediated transport of the CDN.
77. The composition according to clause 72, wherein the CDN transporter-modulating agent is a SLC46A1-binding antibody or a fragment thereof.
78. The composition according to clause 77, wherein the SLC46A1-binding antibody or a fragment thereof targets a first epitope of a SLC46A1 protein that inhibits SLC46A1-mediated transport of the CDN.
79. The composition according to clause 77, wherein the SLC46A1-binding antibody or a fragment thereof targets a second epitope of a SLC46A1 protein that enhances SLC46A1-mediated transport of the CDN.
80. The composition according to clause 72, wherein the CDN transporter-modulating agent is a SLC46A3-binding antibody or a fragment thereof.
81. The composition according to clause 80, wherein the SLC46A3-binding antibody or a fragment thereof targets a first epitope of a SLC46A3 protein that inhibits SLC46A3-mediated transport of the CDN.
82. The composition according to clause 80, wherein the SLC46A3-binding antibody or a fragment thereof targets a second epitope of a SLC46A3 protein that enhances SLC46A3-mediated transport of the CDN.
83. The composition according to clause 72, wherein the CDN transporter-modulating agent is an RNAi agent that modulates expression of SLC19A1, SLC46A1 or SLC46A3 in the cell.
84. A kit comprising:

a first composition comprising a CDN; and

a second composition comprising a CDN transporter-modulating agent.

85. The kit according to clause 84, wherein the CDN is naturally occurring.
86. The kit according to clause 84, wherein the CDN is a non-naturally occurring or synthetic CDN.
87. The kit according to any one of clauses 84-86, wherein the CDN comprises two 2′-5′ phosphodiester linkages.
88. The kit according to any one of clauses 84-86, wherein the CDN comprises two 3′-5′ phosphodiester linkages.
89. The kit according to any one of clauses 43-86, wherein the CDN comprises a 2′-5′ phosphodiester linkage and a 3′-5′ phosphodiester linkage.
90. The kit according to clause 89, wherein the CDN has the formula:

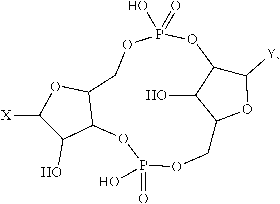

wherein X and Y are each independently:

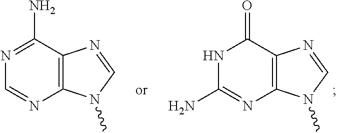

or a salt thereof.
91. The kit according to clause 90, wherein the CDN is cyclic[G(2′5′)pG(3′5′)p].
92. The kit according to clause 90, wherein the CDN is cyclic[A(2′5′)pA(3′5′)p].
93. The kit according to clause 90, wherein the CDN is cyclic[G(2′5′)pA(3′5′)p].
94. The kit according to clause 90, wherein the CDN is cyclic[A(2′5′)pG(3′5′)p].
95. The kit according to any one of clauses 84-94, wherein the CDN transporter-modulating agent modulates a transporter of folate or a folate derivative.
96. The kit according to any one of clauses 84-95, wherein the CDN transporter-modulating agent modulates SLC19A1.
97. The kit according to any one of clauses 84-95, wherein the CDN transporter-modulating agent modulates SLC46A3.
98. The kit according to any one of clauses 95-97, wherein, the CDN transporter-modulating agent is selected from small molecule, organic anion, inorganic anion, an antibody or fragment thereof and an RNAi agent.
99. The kit according to clause 98, wherein the CDN transporter-modulating agent is sulfasalazine or a salt thereof.
100. The kit according to clause 98, wherein the CDN transporter-modulating agent is a SLC19A1-binding antibody or a fragment thereof.
101. The kit according to clause 100, wherein the SLC19A1-binding antibody or a fragment thereof targets a first epitope of a SLC19A1 protein that inhibits SLC19A1-mediated transport of the CDN.
102. The kit according to clause 100, wherein the SLC19A1-binding antibody or a fragment thereof targets a second epitope of a SLC19A1 protein that enhances SLC19A1-mediated transport of the CDN.
103. The kit according to clause 98, wherein the CDN transporter-modulating agent is a SLC46A1-binding antibody or a fragment thereof.
104. The kit according to clause 103, wherein the SLC46A1-binding antibody or a fragment thereof targets a first epitope of a SLC46A1 protein that inhibits SLC46A1-mediated transport of the CDN.
105. The kit according to clause 103, wherein the SLC46A1-binding antibody or a fragment thereof targets a second epitope of a SLC46A1 protein that enhances SLC46A1-mediated transport of the CDN.
106. The kit according to clause 98, wherein the CDN transporter-modulating agent is a SLC46A3-binding antibody or a fragment thereof.
107. The kit according to clause 106, wherein the SLC46A3-binding antibody or a fragment thereof targets a first epitope of a SLC46A3 protein that inhibits SLC46A3-mediated transport of the CDN.
108. The kit according to clause 106, wherein the SLC46A3-binding antibody or a fragment thereof targets a second epitope of a SLC46A3 protein that enhances SLC46A3-mediated transport of the CDN.
109. The kit according to clause 98, wherein the CDN transporter-modulating agent is an RNAi agent that modulates expression of SLC19A1, SLC46A1 or SLC46A3 in the cell.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of modulating activity of a cyclic dinucleotide (CDN) in a cell, the method comprising:
contacting a cell with:
a CDN; and
an amount of a CDN transporter-modulating agent effective to modulate transport of the CDN into the cell;
to modulate activity of the CDN in the cell.

2. The method according to claim 1, wherein the CDN transporter-modulating agent modulates a transporter of folate or a folate derivative.

3. The method according to claim 1, wherein the CDN transporter-modulating agent modulates: SLC19A1-mediated transport of the CDN into the cell;
SLC46A1-mediated transport of the CDN into the cell and/or SLC46A3-mediated transport of the CDN into the cell.

4. The method according to claim 1, wherein contacting the cell with the amount of the CDN transporter-modulating agent is effective to inhibit transport of the CDN into the cell.

5. The method according to claim 1, wherein contacting the cell with the amount of the CDN transporter-modulating agent is effective to enhance transport of the CDN into the cell.

6. The method according to claim 1, wherein the cell is in vitro or
in vivo.

7. The method according to claim 6, wherein the contacting comprises administration of the CDN transporter-modulating agent to a subject.

8. The method according to claim 1, wherein the contacting comprises contacting the cell with composition comprising a CDN.

9. The method according to claim 1, wherein the CDN is a naturally occurring CDN or a non-naturally occurring or synthetic CDN.

10. The method according to claim 1, wherein the CDN comprises two 2′-5′ phosphodiester linkages.

11. The method according to claim 1, wherein the CDN comprises a 2′-5′ phosphodiester linkage and a 3′-5′ phosphodiester linkage.

12. The method according to claim 11, wherein the CDN has the formula: 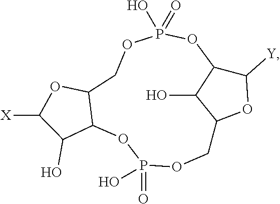
wherein X and Y are each independently: 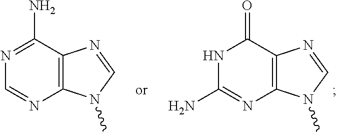
or a salt thereof.

13. The method according to claim 1, wherein the CDN transporter-modulating agent is selected from small molecule, organic anion, inorganic anion, an antibody or fragment thereof and an RNAi agent.

14. The method according to claim 1, wherein the CDN transporter-modulating agent is sulfasalazine or a salt thereof.

15. A composition or kit comprising:
a CDN; and
a CDN transporter-modulating agent.
